(19) United States
(12) Patent Application Publication
(10) Pub. No.: US 2019/0083508 A1
(43) Pub. Date: Mar. 21, 2019

(54) USE OF OX1R ANTAGONISTS FOR THE TREATMENT OF INFLAMMATORY BOWEL DISEASES

(71) Applicant: Inserm (Institut National De La Sante et de la Recherche Medicale), Paris (FR)

(72) Inventors: Alain COUVINEAU, Paris Cedex 18 (FR); Thierry VOISIN, Paris (FR); Eric OGIER-DENIS, Paris (FR); Nassima MESSAL, Paris Cedex 18 (FR); Valérie GRATIO, Paris Cedex 18 (FR); Pascal NICOLE, Paris Cedex 18 (FR)

(21) Appl. No.: 15/528,352

(22) PCT Filed: Nov. 23, 2015
(86) PCT No.: PCT/EP2015/077382

§ 371 Date: May 19, 2017

(30) Foreign Application Priority Data
Nov. 24, 2014 (EP) 14306866.6

(51) Int. Cl. A61K 31/551; A61K 31/472; A61K 31/4706; A61K 45/06; A61P 1/00

(52) U.S. Cl. CPC A61K 31/551; A61K 31/472; A61P 1/00; A61K 45/06; A61K 31/4706

(57) ABSTRACT

The present invention relates to methods and pharmaceutical compositions for the treatment of inflammatory bowel diseases.

### FIELD OF THE INVENTION

The present invention relates to methods and pharmaceutical compositions for the treatment of inflammatory bowel diseases.

### BACKGROUND OF THE INVENTION

Inflammatory bowel disease (IBD), including Crohn's disease (CD) and UC affects approximately 1.5 million Europeans, and its peak onset is in persons with 15 to 30 years of age. IBD incidence is still increasing in developing countries. In France, 64,399 UC patients were estimated from 2008 health security agency records. UC involves the rectum and may affect part of the colon or the entire colon (pancolitis) in an uninterrupted pattern. Changes in diet, antibiotic use, environmental risk factors, and intestinal microflora are involved in IBD pathogenesis and have contributed to the increased prevalence of IBD during the past century.

Despite tremendous advances in our understanding of the pathobiology of IBD, current treatments are not efficient to control the disease over the long term. Indeed, 15-25% of patients with UC are still operated on (total colectomy) and the most powerful treatments (anti-TNF antibodies) provide complete remission at one year of treatment in only 30% of the patients enrolled in clinical trials. Moreover, UC increases the risk of colorectal cancer (18% at 30 years). Recently, the estimated annual per-patient direct medical costs of UC ranged from $6217 to $11 477 in the USA and from €8949 to €10 395 in Europe. Hospitalizations accounted for 41-55% of direct medical costs. The indirect costs accounted for approximately one-third of total costs in the United States and 54-68% in Europe. The total economic burden of UC was estimated at $8.1-14.9 billion annually in the USA and at €12.5-29.1 billion in Europe. Total direct costs were $3.4-8.6 billion in the USA and €5.4-12.6 billion in Europe. Direct costs, hospitalizations and surgeries increased with worsening disease severity. UC is a costly disease and hospitalizations contribute significantly to direct medical costs, and indirect costs are considerable. A better medical control of the disease is required. So far, current treatments are empirically used in UC. They mainly target the immune system and inflammation (aminosalicylates, steroids, and immunomodulators), while new treatments targeting epithelial abnormalities (i.e. ER stress, oxidative stress) will probably be more effective (and better tolerated), to maintain a prolonged remission or even to cure UC. Thus, drugs that prevent primary epithelial defects are mandatory to better manage UC, improve patient's quality of life, avoid terminal complications and considerably reduce the costs of care of UC.

### SUMMARY OF THE INVENTION

The present invention relates to methods and pharmaceutical compositions for the treatment of inflammatory bowel diseases. In particular, the present invention is defined by the claims.

### DETAILED DESCRIPTION OF THE INVENTION

An object of the present invention relates to a method of treating an inflammatory bowel disease in a subject in need thereof comprising administering the subject with a therapeutically effective amount of at least one OX1R antagonist.

As used herein, the term “subject” denotes a mammal, such as a rodent, a feline, a canine, and a primate. Preferably, a subject according to the invention is a human.

As used herein the term “inflammatory bowel disease” has its general meaning in the art and refers to any inflammatory disease that affects the bowel. The term includes but is not limited to ulcerative colitis, Crohn's disease, especially Crohn's disease in a state that affect specifically the colon with or without ileitis, microscopic colitis (lymphocytic colitis and collagenous colitis), infectious colitis caused by bacteria or by virus, radiation colitis, ischemic colitis, pediatric colitis, undetermined colitis, and functional bowel disorders (described symptoms without evident anatomical abnormalities). In some embodiments, the method of the invention is particularly suitable for the treatment of ulcerative colitis.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. The term “treatment” encompasses the prophylactic treatment. As used herein, the term “prevent” refers to the reduction in the risk of acquiring or developing a given condition, or the reduction or inhibition of the recurrence or said condition in a subject who is not ill, but who has been or may be near a subject with the disease.

As used herein, the term “OX1R” has its general meaning in the art and refers to the 7-transmembrane spanning receptor OX1R for orexins.

As used herein the term “OX1R antagonist” has its general meaning in the art and refers to any compound that is able to inhibit the calcium-dependent signalling pathway induced by Orexin. It is known that binding of the orexin to its receptor triggers an influx of calcium, which is coupled to activation of Erk. The receptors also couple to a phospholipase C (PLC)-mediated pathway that releases intracellular calcium stores. The utility of the compounds in accordance with the present invention as orexin receptor OX1R antagonists may be readily determined without undue experimentation by methodology well known in the art, including the “FLIPR Ca2+ Flux Assay” (Okumura et al, Biochem. Biophys. Res. Comm. 280:976-981, 2001). In a typical experiment the OX1 receptor antagonistic activity of the compounds of the present invention was determined in accordance with the following experimental method. For intracellular calcium measurements, Chinese hamster ovary (CHO) cells expressing the rat orexin-1 receptor are grown in Iscove's modified DMEM containing 2 mM L-glutamine, 0.5 g/ml G418, 1% hypoxanthine-thymidine supplement, 100 U/ml penicillin, 100 ug/ml streptomycin and 10% heat-inactivated fetal calf serum (FCS). The cells are seeded at 20,000 cells/well into Becton-Dickinson black 384-well clear bottom sterile plates coated with poly-D-lysine. All reagents were from GIBCO-Invitrogen Corp. The seeded plates are incubated overnight at 37° C. and 5% C02. Ala-6,12 human orexin-A as the agonist is prepared as a 1 mM stock solution in 1% bovine serum albumin (BSA) and diluted in assay buffer (HBSS containing 20 mM HEPES, 0.1% BSA and 2.5 mM probenecid, pH7.4) for use in the assay at a final concentration of 70 pM. Test compounds are prepared as 10 mM stock solution in DMSO, then diluted in 384-well plates, first in DMSO, then assay buffer. On the day of the assay, cells are washed 3 times with 100 ul assay buffer and then incubated for 60 min (37° C., 5% C02) in 60 ul assay buffer containing 1 uM Fluo-4AM ester, 0.02% pluronic acid, and 1% BSA. The dye loading solution is then aspirated and cells are washed 3 times with 100 ul assay buffer. 30 ul of that same buffer is left in each well. Within the Fluorescent Imaging Plate Reader (FLIPR, Molecular Devices), test compounds are added to the plate in a volume of 25 ul, incubated for 5 min and finally 25 ul of agonist is added. Fluorescence is measured for each well at 1 second intervals for 5 minutes and the height of each fluorescence peak is compared to the height of the fluorescence peak induced by 70 pM Ala-6,12 orexin-A with buffer in place of antagonist. For each antagonist, IC50 value (the concentration of compound needed to inhibit 50% of the agonist response) is determined. Alternatively, compound potency can be assessed by a radioligand binding assay (described in Bergman et. al. Bioorg. Med. Chem. Lett. 2008, 18, 1425-1430) in which the inhibition constant is determined in membranes prepared from CHO cells expressing the OX1 receptor. The intrinsic orexin receptor antagonist activity of a compound which may be used in the present invention may be determined by these assays.

OX1R antagonists are well known to the skilled person who may easily identify such antagonists from the following literature:
Boss C, Roch-Brisbare C, Steiner M A, Treiber A, Dietrich H, Jenck F, von Raumer M, Sifferlen T, Brotschi C, Heidmann B, Williams J T, Aissaoui H, Siegrist R, Gatfield J. Structure-Activity Relationship, Biological, and Pharmacological Characterization of the Proline Sulfonamide ACT-462206: a Potent, Brain-Penetrant Dual Orexin1/Orexin2 Receptor Antagonist. ChemMedChem. 2014 Aug. 21.
Christopher J A. Orexin receptor antagonists. Pharm Pat Anal. 2012 July; 1(3):329-46.
Coleman P J, Schreier J D, Cox C D, Breslin M J, Whitman D B, Bogusky M J, McGaughey G B, Bednar R A, Lemaire W, Doran S M, Fox S V, Garson S L, Gotter A L, Harrell C M, Reiss D R, Cabalu T D, Cui D, Prueksaritanont T, Stevens J, Tannenbaum P L, Ball R G, Stellabott J, Young S D, Hartman G D, Winrow C J, Renger J J. Discovery of [(2R,5R)-5-{[(5-fluoropyridin-2-yl)oxy]methyl}-2-methylpiperidin-1-yl][5-methyl-2-(pyrimidin-2-yl)phenyl]methanone (MK-6096): a dual orexin receptor antagonist with potent sleep-promoting properties. ChemMedChem. 2012 Mar. 5; 7(3):415-24, 337.
Cox C D, Breslin M J, Whitman D B, Schreier J D, McGaughey G B, Bogusky M J, Roecker A J, Mercer S P, Bednar R A, Lemaire W, Bruno J G, Reiss D R, Harrell C M, Murphy K L, Garson S L, Doran S M, Prucksaritanont T, Anderson W B, Tang C, Roller S, Cabalu T D, Cui D, Haitinan G D, Young S D, Koblan K S, Winrow C J, Renger J J, Coleman P J. Discovery of the dual orexin receptor antagonist [(7R)-4-(5-chloro-1,3-benzoxazol-2-yl)-7-methyl-1,4-diazepan-1-yl][5-methyl-2-(2H-1,2,3-triazol-2-yl)phenyl]methanone (MK-4305) for the treatment of insomnia. J Med Chem. 2010 Jul. 22; 53(14):5320-32.
Jiaqiang Cai, Fiona E Cooke, Bradley S Sherborne Antagonists of the orexin receptors Expert Opinion on Therapeutic Patents May 2006, Vol. 16, No. 5, Pages 631-646: 631-646.
John A Christopher, Miles S Congreve Treatment and prevention of various therapeutic conditions using OX receptor antagonistic activity (WO2012081692) Expert Opinion on Therapeutic Patents February 2013, Vol. 23, No. 2, Pages 273-277: 273-277.
Langmead C J, Jerman J C, Brough S J, Scott C, Porter R A, Herdon H J. Characterisation of the binding of [3H]-SB-674042, a novel nonpeptide antagonist, to the human orexin-1 receptor. Br J Pharmacol. 2004 January; 141(2):340-6. Epub 2003 Dec. 22.
Paul J Coleman, John J Renger Orexin receptor antagonists: a review of promising compounds patented since 2006 Expert Opinion on Therapeutic Patents March 2010, Vol. 20, No. 3, Pages 307-324: 307-324.
Perrey D A, German N A, Gilmour B P, Li J X, Harris D L, Thomas B F, Zhang Y. Substituted tetrahydroisoquinolines as selective antagonists for the orexin 1 receptor. J Med Chem. 2013 Sep. 12; 56(17):6901-16.
Perrey D A, Gilmour B P, Runyon S P, Thomas B F, Zhang Y. Diaryl urea analogues of SB-334867 as orexin-1 receptor antagonists. Bioorg Med Chem Lett. 2011 May 15; 21(10):2980-5.
Porter R A, Chan W N, Coulton S, Johns A, Hadley M S, Widdowson K, Jerman J C, Brough S J, Coldwell M, Smart D, Jewitt F, Jeffrey P, Austin N. 1,3-Biarylureas as selective non-peptide antagonists of the orexin-1 receptor. Bioorg Med Chem Lett. 2001 Jul. 23; 11(14):1907-10.
Roecker A J, Coleman P J (2008). “Orexin receptor antagonists: medicinal chemistry and therapeutic potential”. Curr Top Med Chem 8 (11): 977-87.
Roecker A J, Coleman P J. Orexin receptor antagonists: medicinal chemistry and therapeutic potential. Curr Top Med Chem. 2008; 8(11):977-87.
Roecker A J, Mercer S P, Harrell C M, Garson S L, Fox S V, Gotter A L, Prueksaritanont T, Cabalu T D, Cui D, Lemaire W, Winrow C J, Renger J J, Coleman P J. Discovery of dual orexin receptor antagonists with rat sleep efficacy enabled by expansion of the acetonitrile-assisted/diphosgene-mediated 2,4-dichloropyrimidine synthesis. Bioorg Med Chem Lett. 2014 May 1; 24(9):2079-85.
Smart D, Sabido-David C, Brough S J, Jewitt F, Johns A, Porter R A, Jerman J C. SB-334867-A: the first selective orexin-1 receptor antagonist. Br J Pharmacol. 2001 March; 132(6):1179-82.
Whitman D B, Cox C D, Breslin M J, Brashear K M, Schreier J D, Bogusky M J, Bednar R A, Lemaire W, Bruno J G, Hartman G D, Reiss D R, Harrell C M, Kraus R L, Li Y, Garson S L, Doran S M, Prueksaritanont T, Li C, Winrow C J, Koblan K S, Renger J J, Coleman P J. Discovery of a potent, CNS-penetrant orexin receptor antagonist based on an n,n-disubstituted-1,4-diazepane scaffold that promotes sleep in rats. ChemMedChem. 2009 July; 4(7):1069-74.
Yoshida Y, Terauchi T, Naoe Y, Kazuta Y, Ozaki F, Beuckmann C T, Nakagawa M, Suzuki M, Kushida I, Takenaka O, Ueno T, Yonaga M. Design, synthesis, and structure-activity relationships of a series of novel N-aryl-2-phenylcyclopropanecarboxamide that are potent and orally active orexin receptor antagonists. Bioorg Med Chem. 2014 Sep. 8. pii: S0968-0896(14)00630-0.

Other examples of OX1R antagonists are also described in the following patent publications:
EP0849361
US20080132490
US20090163485
U.S. Pat. No. 6,309,854
WO 2014099698
WO00047576
WO00047577
WO00047580
WO01000787
WO01068609
WO01085693
WO01096302
WO02044172
WO02051232
WO02051838
WO02089800
WO02090355
WO03002559
WO03002561
WO03032991
WO03037847
WO03041711
WO03051368
WO03051871
WO03051872
WO03051873
WO19909024
WO19958533
WO2003002561
WO2003051872
WO2004004733
WO2004026866
WO2004033418
WO2004041791
WO2004041807
WO2004041816
WO2004052876
WO2004085403
WO2004096780
WO2005060959
WO2005075458
WO2005118548
WO2005118548
WO2006067224
WO2006110626
WO2006127550
WO2007019234
WO2007025069
WO2007061763
WO2007085718
WO2007122591
WO2007126934
WO2007126935
WO2008008517
WO2008008518
WO2008008551
WO2008020405
WO2008026149
WO2008038251
WO2008065626
WO2008078291
WO2008081399
WO2008107335
WO2008110488
WO2008117241
WO2008143856
WO2008147518
WO2008150364
WO2009003993
WO2009003997
WO2009004585
WO2009011775
WO2009016087
WO2009016564
WO2009020642
WO2009022311
WO2009034133
WO2009058238
WO2009079637
WO2011053522
WO2013059163
WO2013059222
WO2013062857
WO2013062858
WO2014066196
WO2014085208
WO2014099696
and WO2014099697.

In some embodiments, the OX1R antagonist of the present invention is SB408124 which is:

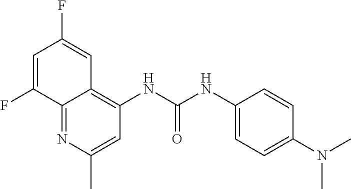 [SMILES: Cc1cc(NC(=O)Nc2ccc(N(C)C)cc2)c2cc(F)cc(F)c2n1]

In some embodiments, the OX1R antagonist of the present invention is selected from the group consisting of:

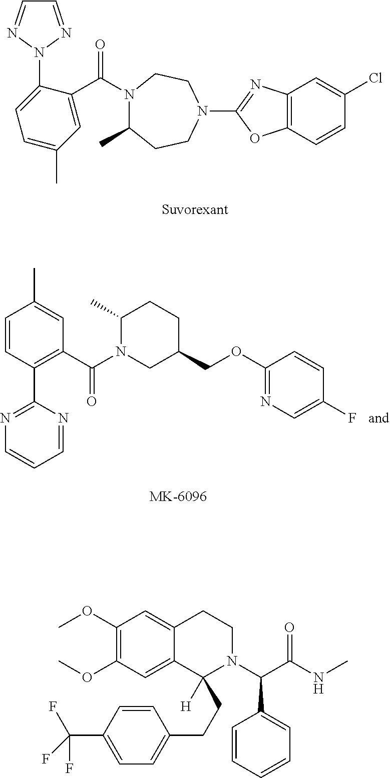 [SMILES: Cc1ccc(-n2nccn2)c(C(=O)N2CCN(c3nc4cc(Cl)ccc4o3)CC[C@H]2C)c1 ; Cc1ccc(-c2ncccn2)c(C(=O)N2C[C@H](COc3ccc(F)cn3)CC[C@H]2C)c1 ; CNC(=O)[C@@H](c1ccccc1)N1CCc2cc(OC)c(OC)cc2[C@@H]1CCc1ccc(C(F)(F)F)cc1]

In some embodiments, the OX1R antagonist of the present invention is selected from the group consisting of:

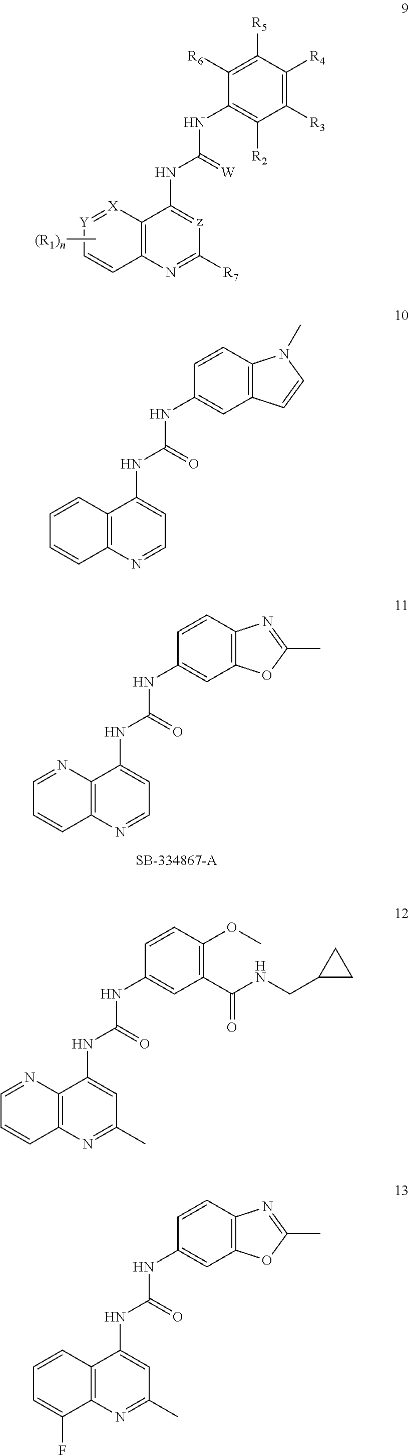

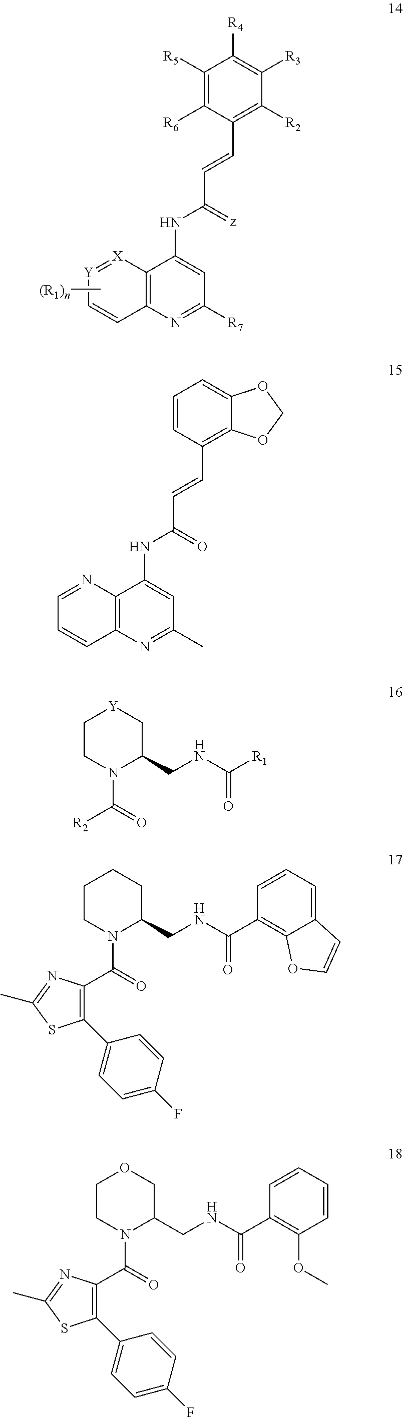

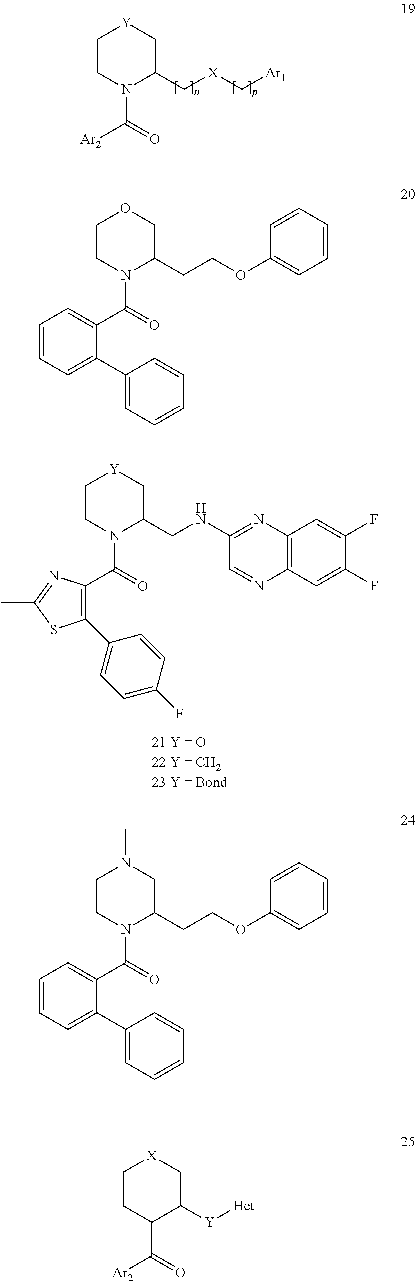

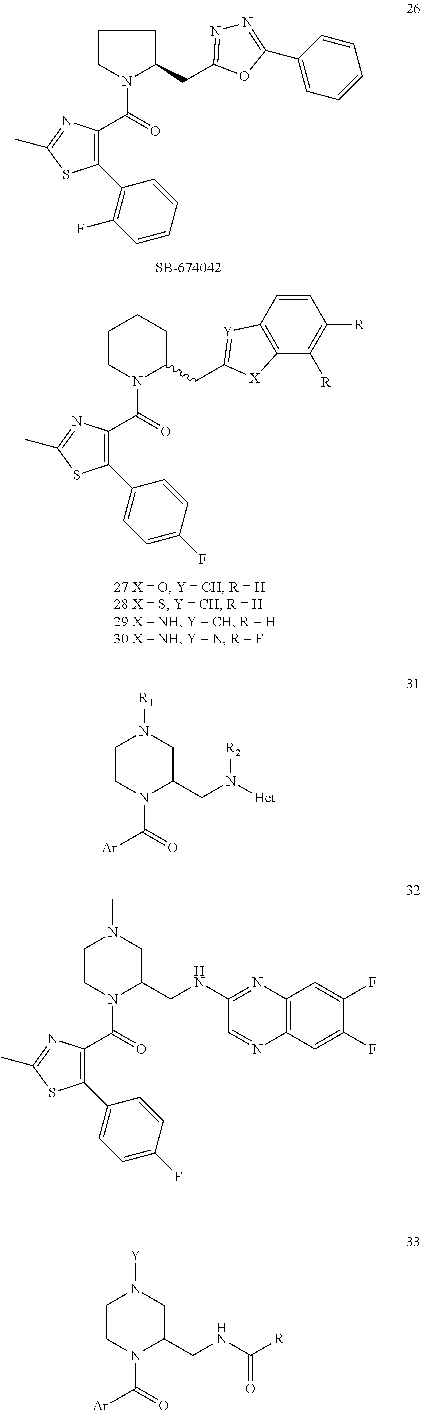

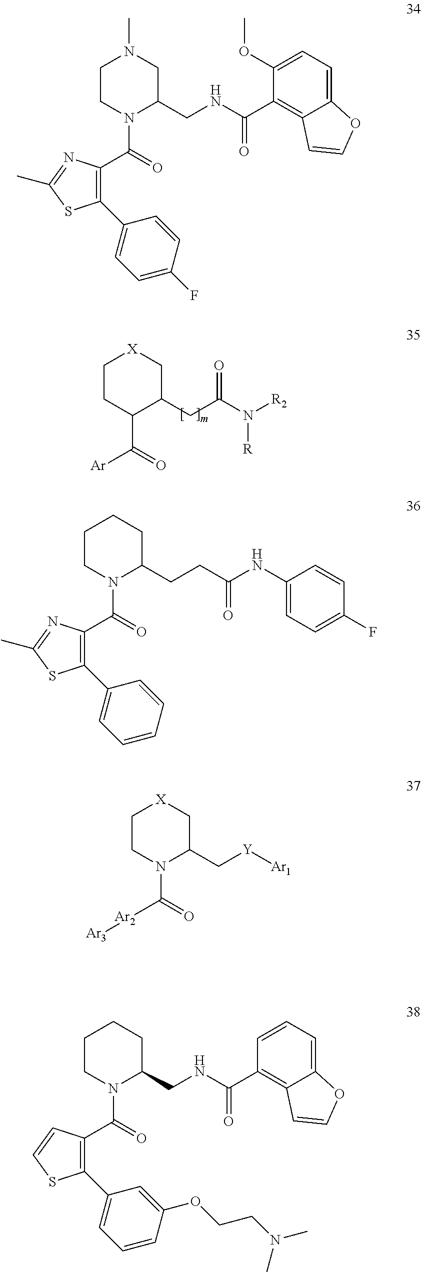

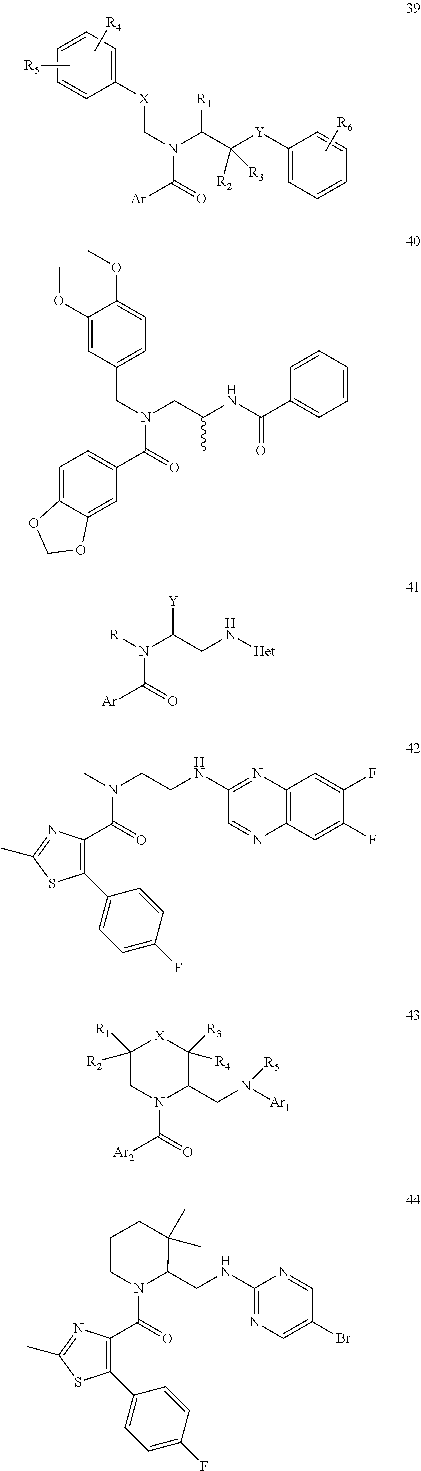

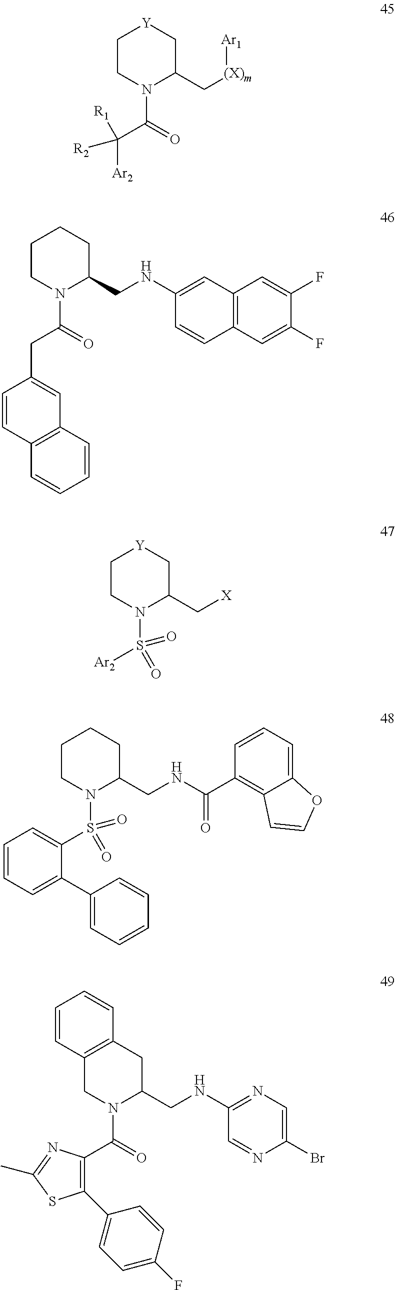

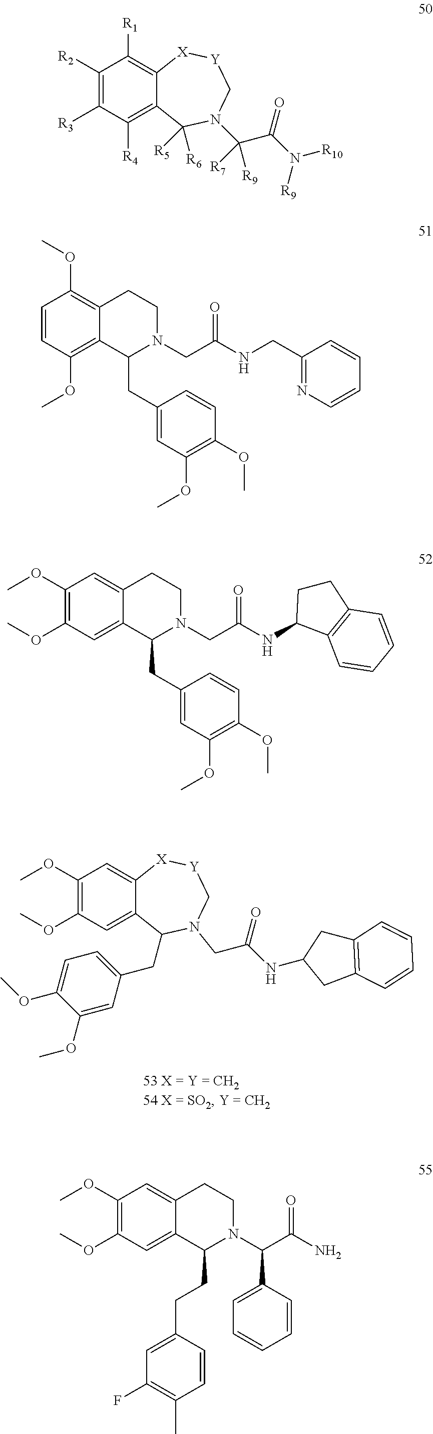

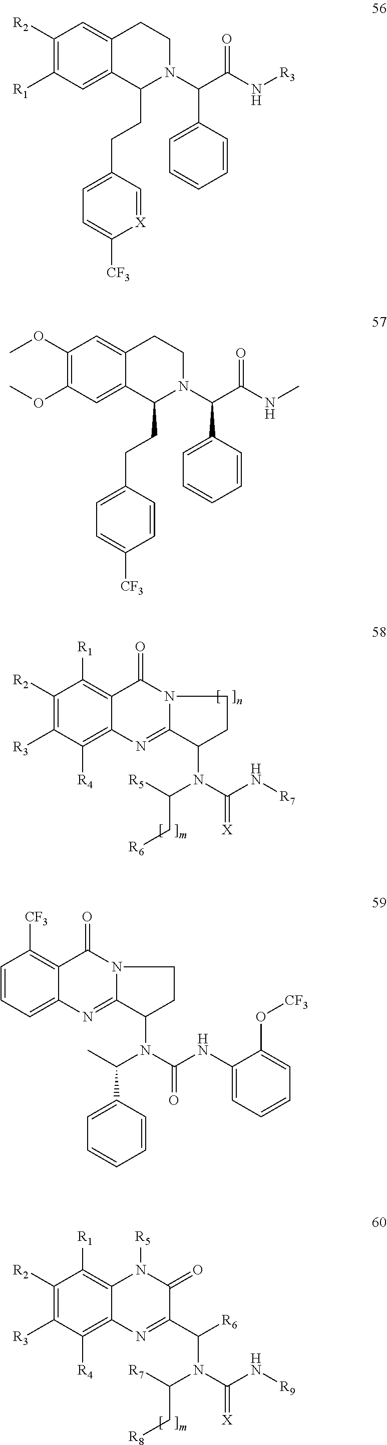

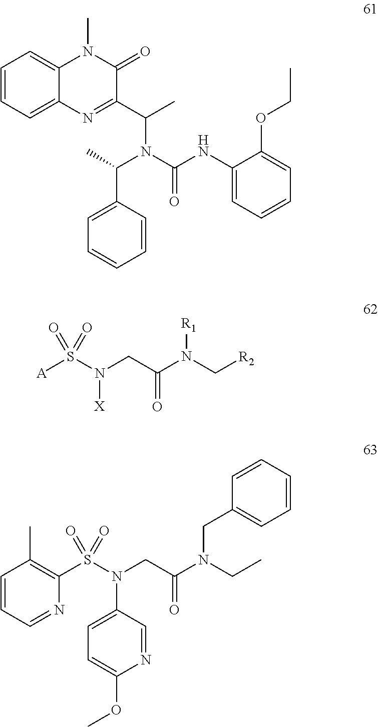

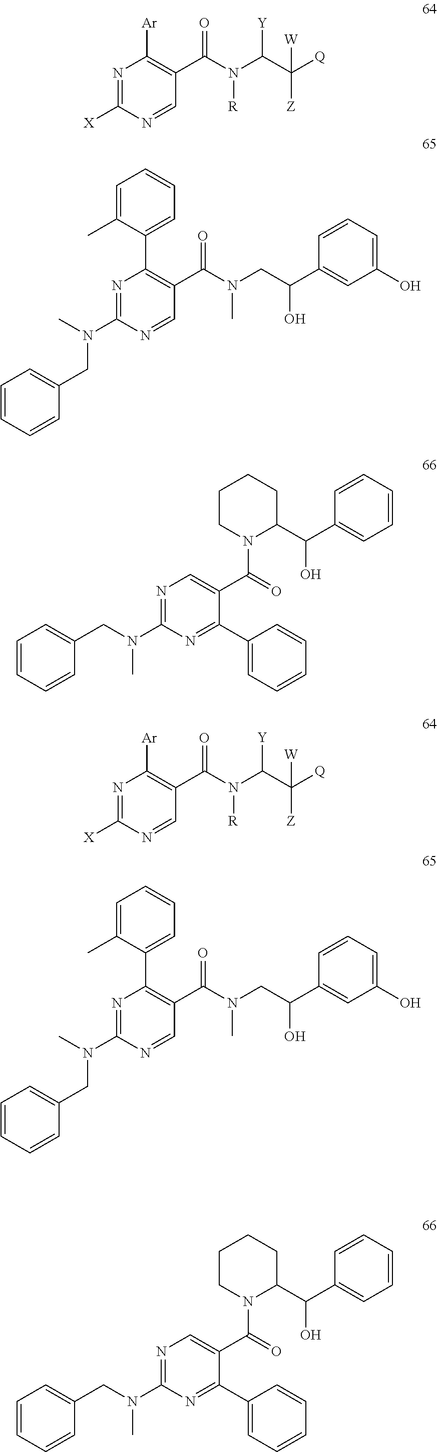

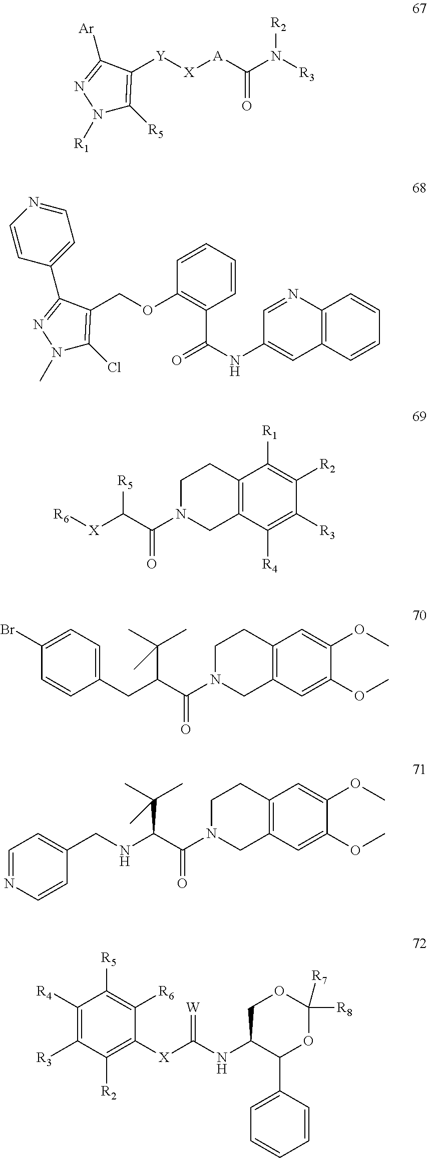

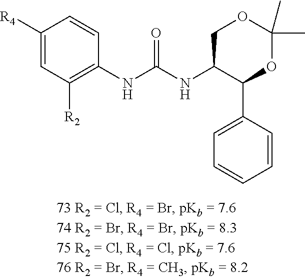

In some embodiments, the OX1R antagonist of the present invention is selected from the group consisting of:

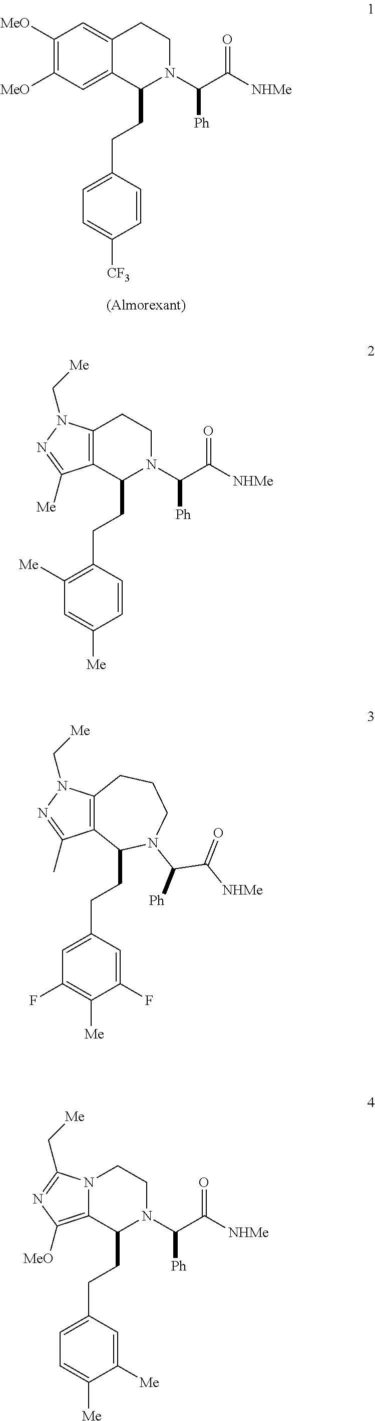 [SMILES: CNC(=O)[C@@H](c1ccccc1)N1CCc2cc(OC)c(OC)cc2[C@@H]1CCc1ccc(C(F)(F)F)cc1 ; CCn1nc(C)c2c1CCN([C@@H](C(=O)NC)c1ccccc1)[C@H]2CCc1ccc(C)cc1C ; CCn1nc(C)c2c1CCCN([C@@H](C(=O)NC)c1ccccc1)[C@H]2CCc1cc(F)c(C)c(F)c1 ; CCc1nc(OC)c2n1CCN([C@@H](C(=O)NC)c1ccccc1)[C@H]2CCc1ccc(C)c(C)c1]

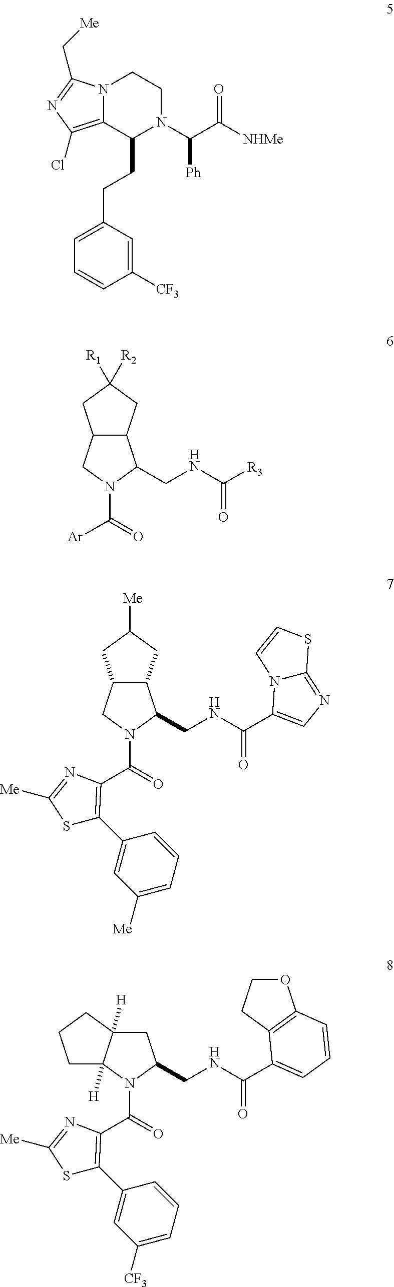

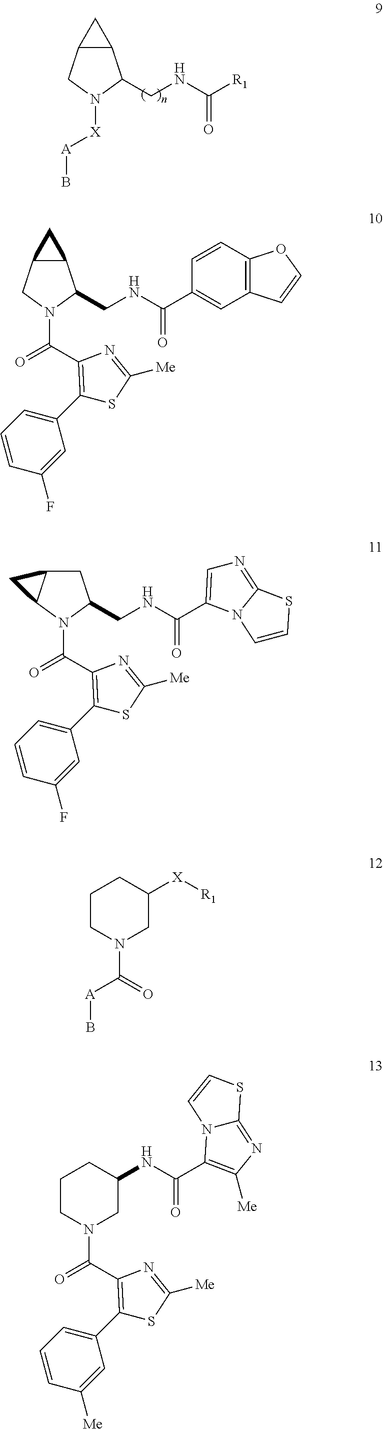

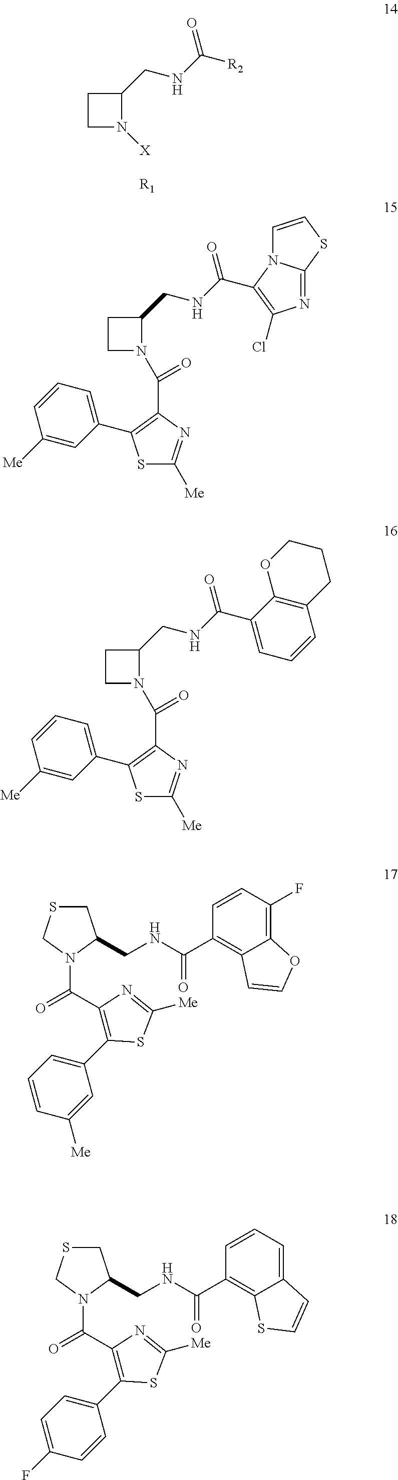

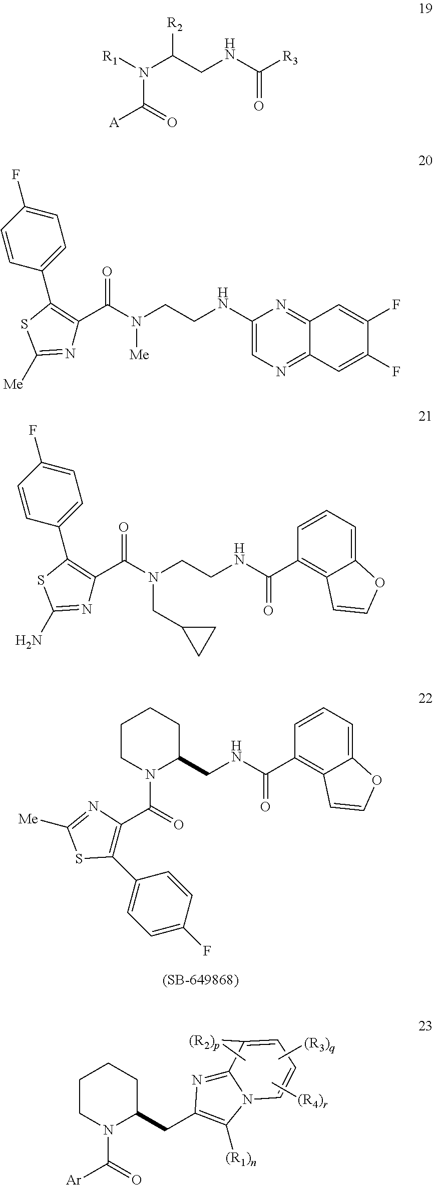

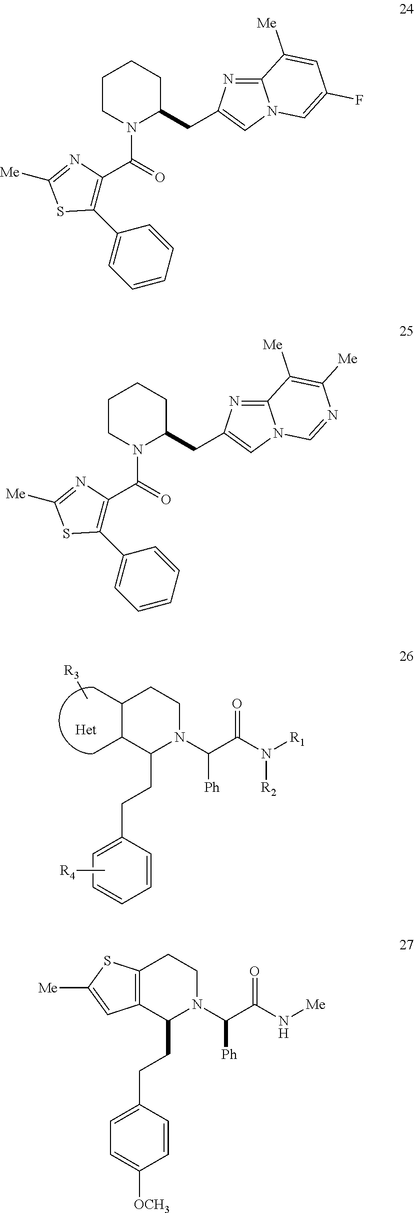

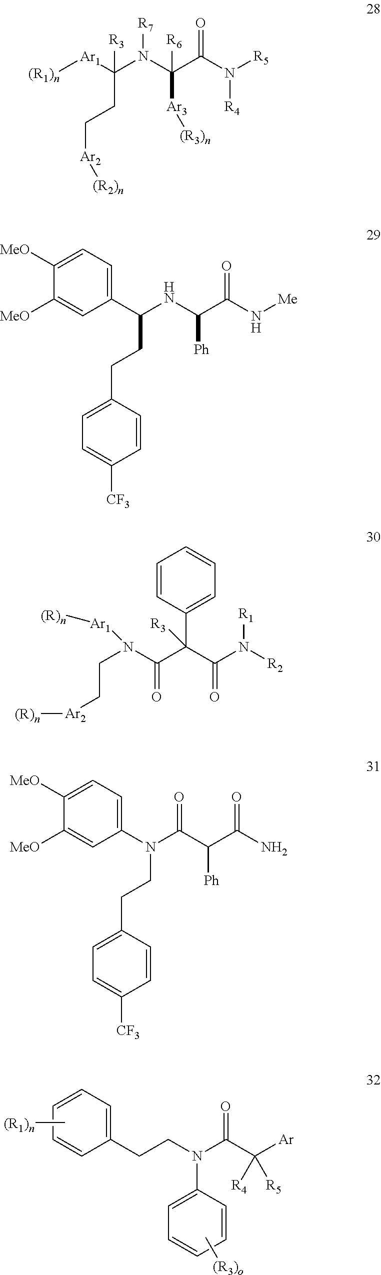

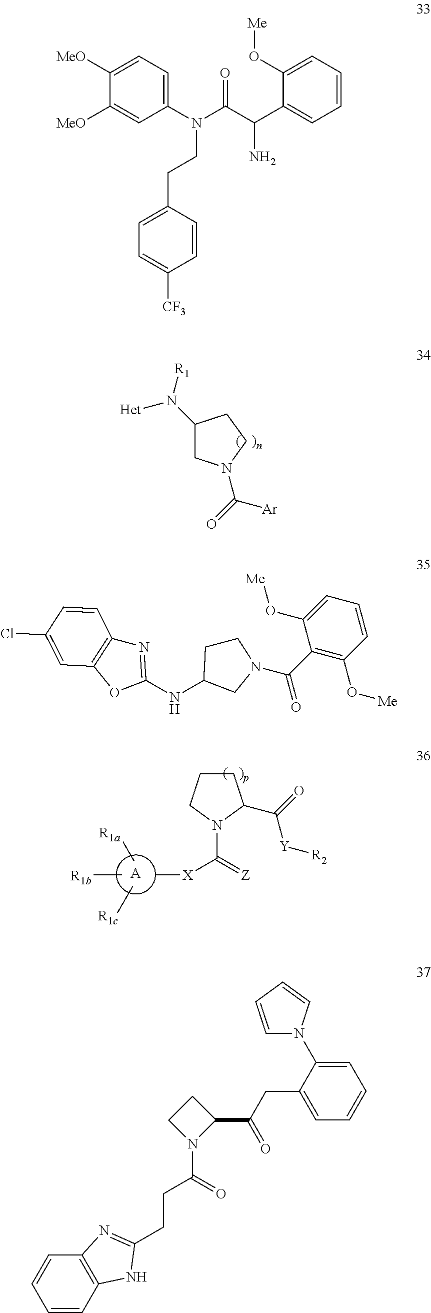

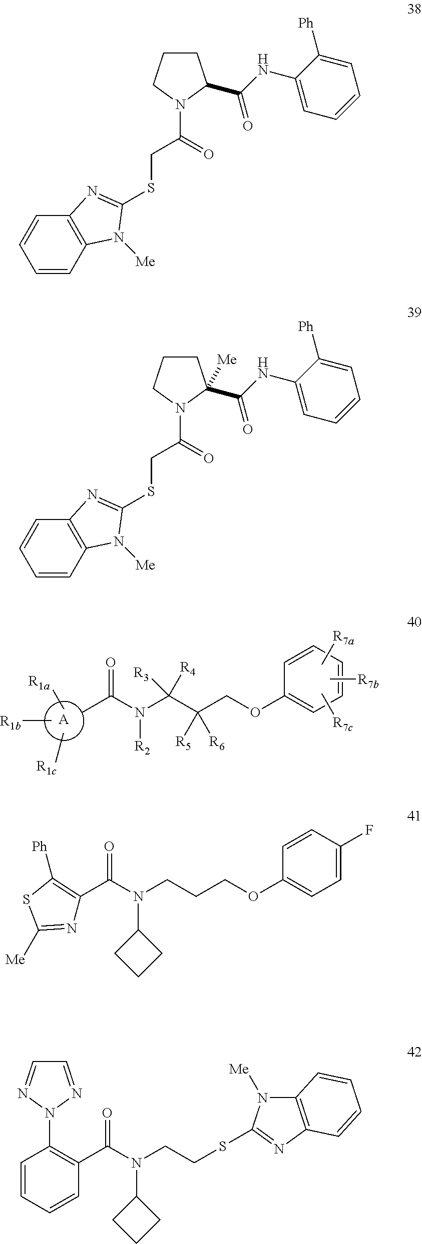

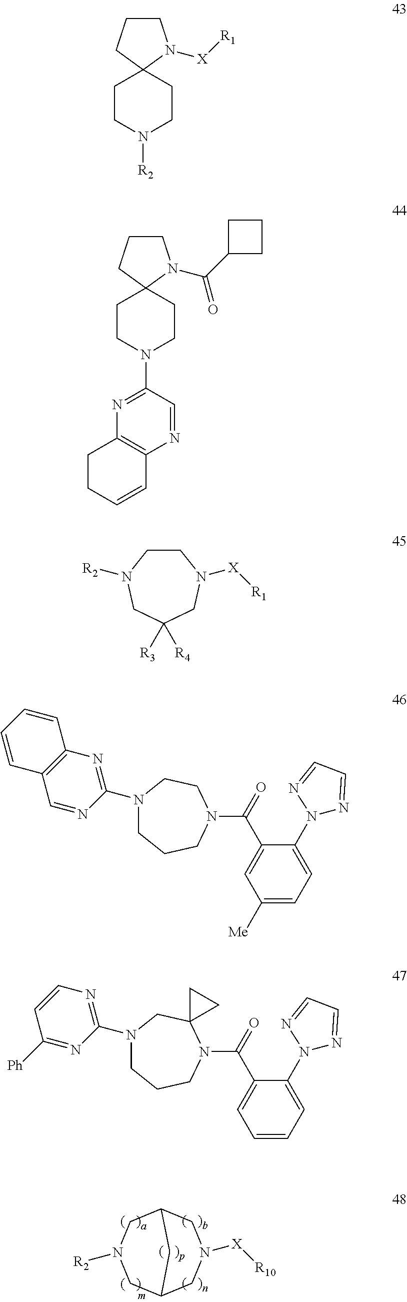

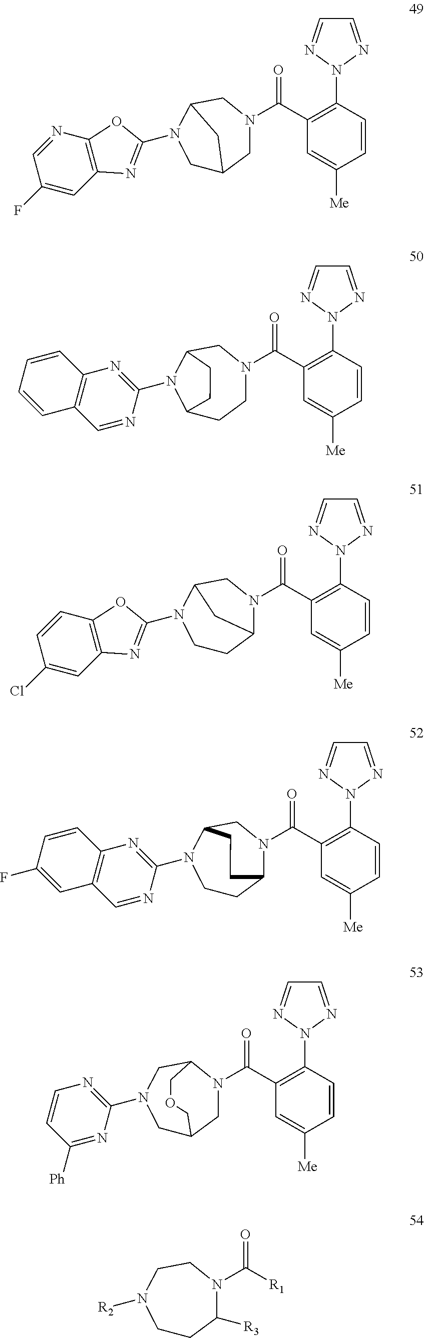

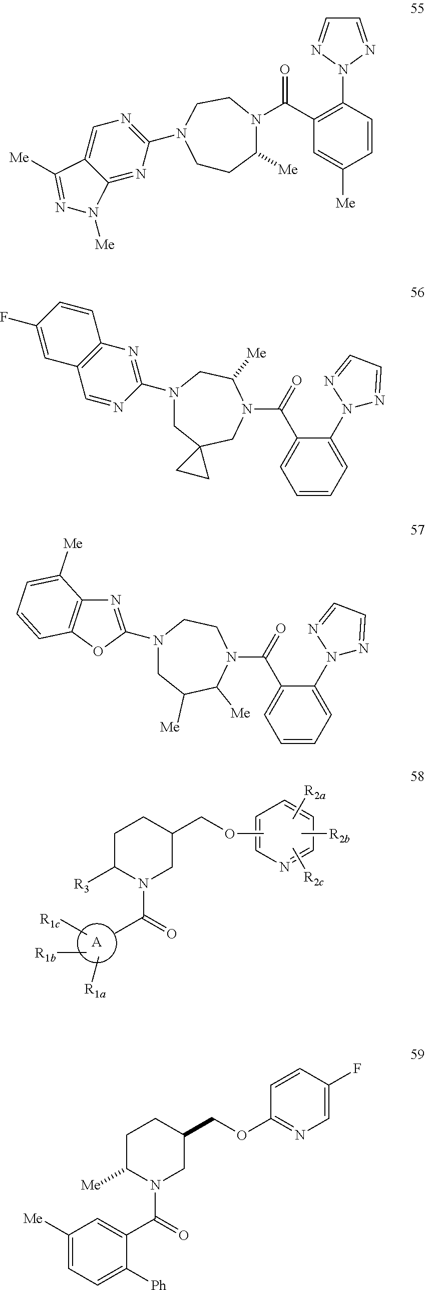

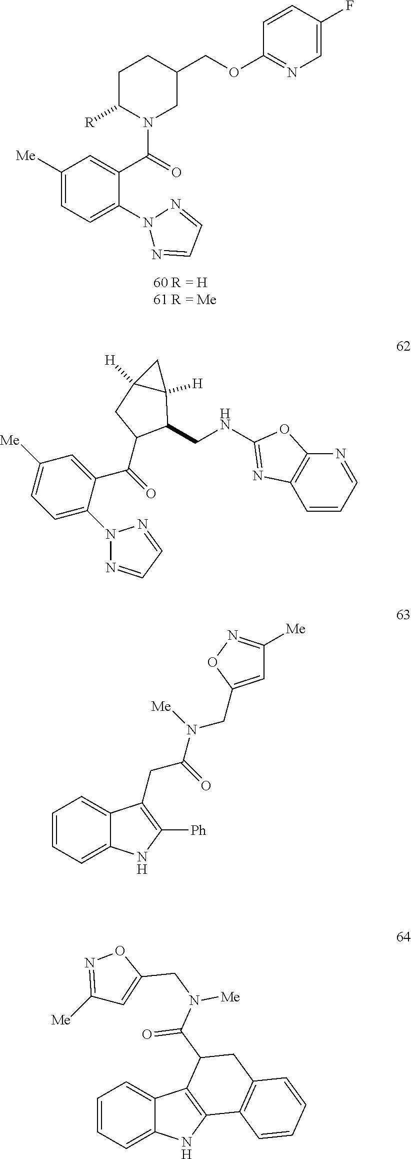

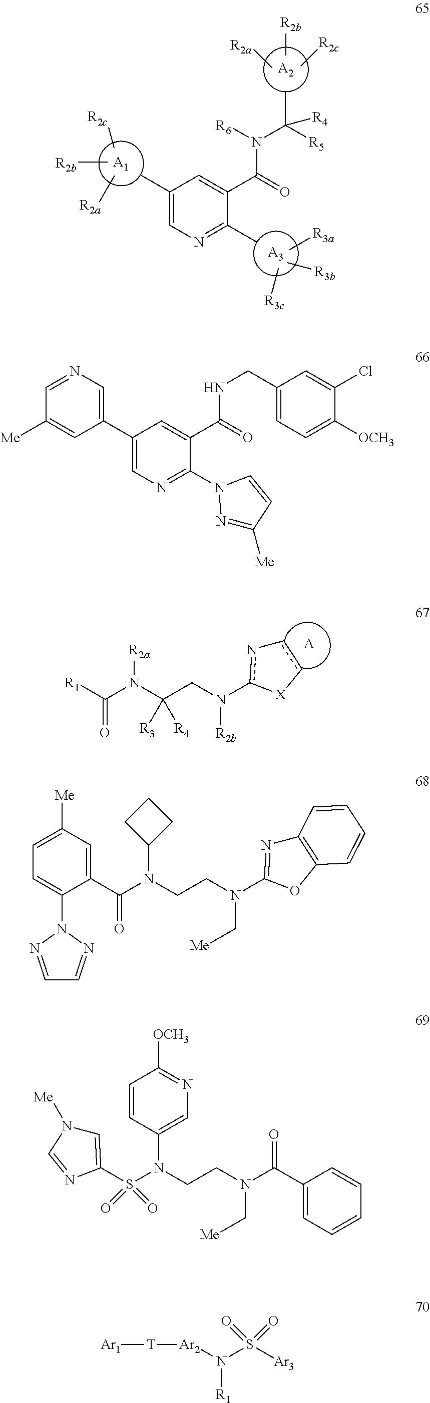

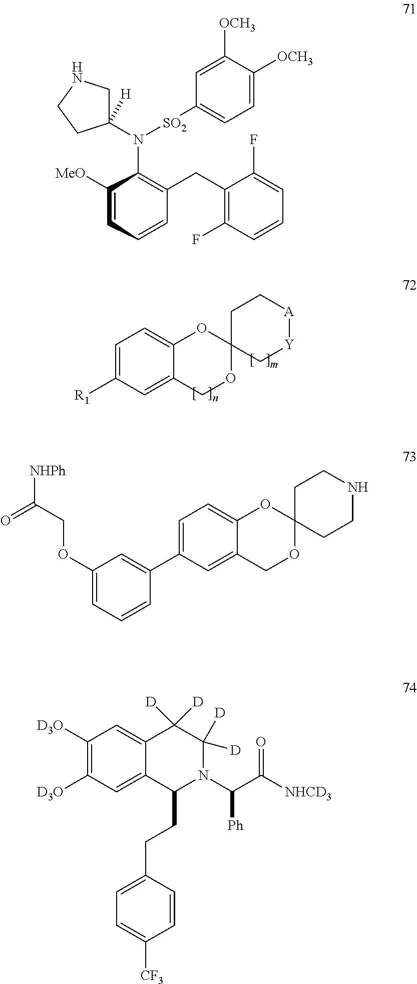

In some embodiments, the OX1R antagonist of the present invention is selected from 2-pyridyloxy-3-substituted-4-nitrile orexin receptor antagonists that are disclosed in WO 2014066196. In some embodiments, the OX1R antagonist of the present invention is selected from the group consisting of:
3-methyl-2-{[(3R,6R)-6-methyl-1-{[2-(2H-1,2,3-triazol-2-1)phenyl]carbonyl}piperidin-3-yl]oxy}pyridine-4-carbonitrile;
2-{[(3R,6R)-1-{[6-methoxy-2-(2H-1,2,3-triazol-2-yl)pyridin-3-yl]carbonyl}-6-methylpiperidin-3-yl]oxy}-3-methylpyridine-4-carbonitrile;
2-{[(3R,6R)-1-{[5-chloro-2-(2H-1,2,3-triazol-2-yl)phenyl]carbonyl}-6-ethylpiperidin-3-yl]oxy}-3-methylpyridine-4-carbonitrile;
2-{[(3R,6R)-1-{[4-chloro-2-(2H-1,2,3-triazol-2-yl)phenyl]carbonyl}-6-ethylpiperidin-3-yl]oxy}-3-methylpyridine-4-carbonitrile;
2-({(3R,6R)-1-[(2-cyclopropyl-6-methoxypyridin-3-yl)carbonyl]-6-methylpiperidin-3-yl}oxy)-3-methylpyridine-4-carbonitrile;
3-methyl-2-{[(3R,6R)-6-methyl-1-{[5-methyl-2-(2H-1,2,3-triazol-2-yl)phenyl]carbonyl}piperidin-3-yl]oxy}pyridine-4-carbonitrile;
2-{[(3R,6R)-1-{[4-methoxy-2-(2H-1,2,3-triazol-2-yl)phenyl]carbonyl}-6-methylpiperidin-3-yl]oxy}-3-methylpyridine-4-carbonitrile;
3-methyl-2-{[(3R,6R)-6-methyl-1-{[4-methyl-2-(2H-1,2,3-triazol-2-yl)phenyl]carbonyl}piperidin-3-yl]oxy}pyridine-4-carbonitrile;
2-{[(3R,6R)-1-[4-fluoro-2-(2H-1,2,3-triazol-2-yl)phenyl]carbonyl-6-methylpiperidin-3-yl]oxy}-3-methylpyridine-4-carbonitrile;
3-methyl-2-{[(3R,6R)-6-methyl-1-{[4-(2H-1,2,3 riazol-2-yl)pyridin-3-yl]carbonyl}piperidin-3-yl]oxy}pyridine-4-carbonitrile;
2-{[(3R,6R)-1-{[2-fluoro-6-(2H-1,2,3-triazol-2-yl)phenyl]carbonyl}-6-methylpiperidin-3-yl]oxy}-3-methylpyridine-4-carbonitrile;
2-{[(3R,6R)-1-{[3-fluoro-2-(2H-1,2,3-triazol-2-yl)phenyl]carbonyl}-6-methylpiperidin-3-yl]oxy}-3-methylpyridine-4-carbonitrile;
3-methyl-2-{[(3R,6R)-6-methyl-1-{[3-methyl-2-(2H-1,2,3-triazol-2-yl)phenyl]carbonyl}piperidin-3-yl]oxy}pyridine-4-carbonitrile;
2-{[(3R,6R)-1-{[2-methoxy-6-(2H-1,2,3-triazol-2-yl)phenyl]carbonyl}-6-methylpiperidin-3-yl]oxy}-3-methylpyridine-4-carbonitrile;
3-methyl-2-{[(3R,6R)-6-methyl-1-{[2-methyl-6-(2H-1,2,3-triazol-2-yl)phenyl]carbonyl}piperidin-3-yl]oxy}pyridine-4-carbonitrile;
2-({(3R,6R)-1-[(2-cyclopropylphenyl)carbonyl]-6-methylpiperidin-3-yl}oxy)-3-methylpyridine-4-carbonitrile;
3-methyl-2-{[(3R,6R)-6-methyl-1-{[2-(trifluoromethyl)phenyl]carbonyl}piperidin-3-yl]oxy}pyridine-4-carbonitrile;
2-{[(3R,6R)-1-{[2-fluoro-6-(1,3-thiazol-2-yl)phenyl]carbonyl}-6-methylpiperidin-3-yl]oxy}-3-methylpyridine-4-carbonitrile;
2-({(3R,6R)-1-[(2-ethoxyphenyl)carbonyl]-6-methylpiperidin-3-yl}oxy)-3-methylpyridine-4-carbonitrile;
3-methyl-2-{[(3R,6R)-6-methyl-1-[(4-phenylisothiazol-5-yl)carbonyl]piperidin-3-yl]oxy}pyridine-4-carbonitrile;
3-methyl-2-{[(3R,6R)-6-methyl-1-{[2-(1-methylethoxy)phenyl]carbonyl}piperidin-3-yl]oxy}pyridine-4-carbonitrile;
2-{[(3R,6R)-1-{[2-fluoro-6-(1,3-thiazol-4-yl)phenyl]carbonyl}-6-methylpiperidin-3-yl]oxy}-3-methylpyridine-4-carbonitrile;
3-methyl-2-({(3R,6R)-6-methyl-1-[(3-phenylpyridin-4-yl)carbonyl]piperidin-3-yl}oxy)pyridine-4-carbonitrile;
3-methyl-2-{[(3R,6R)-6-methyl-1-{[3-(1,3-thiazol-4-yl)pyridin-2-yl]carbonyl}piperidin-3-yl]oxy}pyridine-4-carbonitrile;
2-{[(3R,6R)-1-{[2-fluoro-5-(1,3-thiazol-5-yl)pyridin-4-yl]carbonyl}-6-methylpiperidin-3-yl]oxy}-3-methylpyridine-4-carbonitrile;
2-{[(3R,6R)-1-{[5-fluoro-2-(1,3-thiazol-4-yl)phenyl]carbonyl}-6-methylpiperidin-3-yl]oxy}-3 methylpyridine-4-carbonitrile;
3-methyl-2-{[(3R,6R)-6-methyl-1-{[2-(1,3-oxazol-2-yl)phenyl]carbonyl}piperidin-3-yl]oxy}pyridine-4-carbonitrile;
2-{[(3R,6R)-1-{[5-fluoro-2-(1,3-thiazol-2-yl)phenyl]carbonyl}-6-methylpiperidin-3-yl]oxy}-3 methylpyridine-4-carbonitrile;
3-methyl-2-({(3R,6R)-6-methyl-1-[(2-pyrrolidin-1-ylphenyl)carbonyl]piperidin-3-yl}oxy)pyridine-4-carbonitrile;
3-methyl-2-({(3R,6R)-6-methyl-1-[(2-phenoxyphenyl)carbonyl]piperidin-3-yl}oxy)pyridine-4-carbonitrile;
3-methyl-2-{[(3R,6R)-6-methyl-1-{[2-(1,3-thiazol-4-yl)thiophen-3-yl]carbonyl}piperidin-3-yl]oxy}pyridine-4-carbonitrile;
3-methyl-2-{[(3R,6R)-6-methyl-1-{[2-(1,3-thiazol-2-yl)thiophen-3-yl]carbonyl}piperidin-3-yl]oxy}pyridine-4-carbonitrile;
3-methyl-2-{[(3R,6R)-6-methyl-1-{[2-(1,3-thiazol-4-yl)phenyl]carbonyl}piperidin-3-yl]oxy}pyridine-4-carbonitrile;
2-{[(3R,6R)-1-{[4-fluoro-2-(1,3-thiazol-2-yl)phenyl]carbonyl}-6-methylpiperidin-3-yl]oxy}-3 methylpyridine-4-carbonitrile;
2-{[(3R,6R)-1-{[5-fluoro-2-(1,3-thiazol-2-yl)pyridin-3-yl]carbonyl}-6-methylpiperidin-3-yl]oxy}-3-methylpyridine-4-carbonitrile;
3-methyl-2-{[(3R,6R)-6-methyl-1-{[2-(1-methyl-1H-pyrazol-4-yl)phenyl]carbonyl}piperidin-3 yl]oxy}pyridine-4-carbonitrile;
3-methyl-2-{[(3R,6R)-6-methyl-1-{[3-(1,3-thiazol-2-yl)thiophen-2-yl]carbonyl}piperidin-3-yl]oxy}pyridine-4-carbonitrile;
3-methyl-2-{[(3R,6R)-6-methyl-1-{[3-(1,3-thiazol-4-yl)thiophen-2-yl]carbonyl}piperidin-3-yl]oxy}pyridine-4-carbonitrile;
3-methyl-2-{[(3R,6R)-6-methyl-1-{[2-(2H-1,2,3-triazol-2-yl)thiophen-3-yl]carbonyl}piperidin 3-yl]oxy}pyridine-4-carbonitrile;
3-methyl-2-{[(3R,6R)-6-methyl-1-{[2-(5-methyl-2H-tetrazol-2-yl)phenyl]carbonyl}piperidin-3 yl]oxy}pyridine-4-carbonitrile;
2-{[(3R,6R)-1-{[5-(4-fluorophenyl)-2-methyl-1,3-thiazol-4-yl]carbonyl}-6-methylpiperidin-3-yl]oxy}-3-methylpyridine-4-carbonitrile;
3-methyl-2-{[(3R,6R)-6-methyl-1-{[2-(5-methyl-1,2,4-oxadiazol-3-yl)phenyl]carbonyl}piperidin-3-yl]oxy}pyridine-4-carbonitrile;
3-methyl-2-{[(3R,6R)-6-methyl-1-{[2-(4-methyl-1H-pyrazol-1-yl)phenyl]carbonyl}piperidin-3-yl]oxy}pyridine-4-carbonitrile;
3-methyl-2-{[(3R,6R)-6-methyl-1-{[2-(1,3-thiazol-2-yl)phenyl]carbonyl}piperidin-3-yl]oxy}pyridine-4-carbonitrile;
3-methyl-2-({(3R,6R)-6-methyl-1-[(1-methyl-3-phenyl-1H-pyrazol-4-yl)carbonyl]piperidin-3-yl}oxy)pyridine-4-carbonitrile;
3-methyl-2-{[(3R,6R)-6-methyl-1-{[1-methyl-3-(1,3-thiazol-2-yl)-1H-pyrazol-4-yl]carbonyl}piperidin-3-yl]oxy}pyridine-4-carbonitrile;
3-methyl-2-({(3R,6R)-6-methyl-1-[(3-methyl-5-phenyl isothiazol-4-yl)carbonyl]piperidin-3-yl}oxy)pyridine-4-carbonitrile;
2-({(3R,6R)-1-[(6-methoxy-2,4′-bipyridin-3-yl)carbonyl]-6-methylpiperidin-3-yl}oxy)-3-methylpyridine-4-carbonitrile;
2-{[(3R,6R)-1-{[2-(6-methoxypyridin-3-yl)phenyl]carbonyl}-6-methylpiperidin-3-yl]oxy}-3-methylpyridine-4-carbonitrile;
3-methyl-24 {(3R,6R)-6-methyl-1-[(4-phenylisothiazol-3-yl)carbonyl]piperidin-3-yl}oxy)pyridine-4-carbonitrile;
2-({(3R,6R)-1-[(2-cyclopropyl-4-methylphenyl)carbonyl]-6-methylpiperidin-3-yl}oxy)-3-methylpyridine-4-carbonitrile;
2-({(3R,6R)-1-[(2-cyclopropyl-4-methoxyphenyl)carbonyl]-6-methylpiperidin-3-yl}oxy)-3-methylpyridine-4-carbonitrile;
2-({(3R,6R)-1-[(2-cyclopropyl-4-fluorophenyl)carbonyl]-6-methylpiperidin-3-yl}oxy)-3-methylpyridine-4-carbonitrile;
2-{[(3R,6R)-1-{[5-(hydroxymethyl)biphenyl-2-yl]carbonyl}-6-methylpiperidin-3-yl]oxy}-3-methylpyridine-4-carbonitrile;
3-methyl-2-{[(3R,6R)-6-methyl-1-{[4-(2H-1,2,3-triazol-2-yl)isothiazol-3-yl]carbonyl}piperidin-3-yl]oxy}pyridine-4-carbonitrile;
2-({(3R,6R)-1-[(5-fluoro-2-pyridin-2-ylphenyl)carbonyl]-6-methylpiperidin-3-yl}oxy)-3-methylpyridine-4-carbonitrile;
3-methyl-2-({(3R,6R)-6-methyl-1-[(2-methyl-5-phenyl-1,3-thiazol-4-yl)carbonyl]piperidin-3-yl}oxy)pyridine-4-carbonitrile;
3-methyl-2-{[(3R,6R)-6-methyl-1-{[2-(3-methyl-1,2,4-oxadiazol-5-yl)phenyl]carbonyl}piperidin-3-yl]oxy}pyridine-4-carbonitrile;
2-({(3R,6R)-1-[(2-ethylphenyl)carbonyl]-6-methylpiperidin-3-yl}oxy)-3-methylpyridine-4-carbonitrile;
3-methyl-2-({(3R,6R)-6-methyl-1-[(2-phenylpyridin-3-yl)carbonyl]piperidin-3-yl}oxy)pyridine-4-carbonitrile;
3-methyl-2-{[(3R,6R)-6-methyl-1-{[2-(methylsulfanyl)phenyl]carbonyl}piperidin-3-yl]oxy}pyridine-4-carbonitrile;
3-methyl-2-{[(3R,6R)-6-methyl-1-{[2-(trifluoromethoxy)phenyl]carbonyl}piperidin-3-yl]oxy}pyridine-4-carbonitrile;
3-methyl-2-({(3R,6R)-6-methyl-1-[(2-pyridin-2-ylcyclopent-1-en-1-yl)carbonyl]piperidin-3-yl}oxy)pyridine-4-carbonitrile;
2-{[(3R,6R)-1-{[4-(fluoromethoxy)-2-(2H-1,2,3-triazol-2-yl)phenyl]carbonyl}-6-methylpiperidin-3-yl]oxy}-3-methylpyridine-4-carbonitrile;
2-{[(3R,6R)-1-{[4-(difluoromethoxy)-2-(2H-1,2,3-triazol-2-yl)phenyl]carbonyl}-6-methylpiperidin-3-yl]oxy}-3-methylpyridine-4-carbonitrile;
2-({(3R,6R)-1-[(2-cyclobutyl-6-methoxypyridin-3-yl)carbonyl]-6-methylpiperidin-3-yl}oxy)-3-methylpyridine-4-carbonitrile;
2-{[(3R,6R)-1-{[6-chloro-4-(2H-1,2,3-triazol-2-yl)pyridin-3-yl]carbonyl}-6-methylpiperidin-3-yl]oxy}-3-methylpyridine-4-carbonitrile;
2-({(3R,6R)-1-[(2-ethoxypyridin-3-yl)carbonyl]-6-methylpiperidin-3-yl}oxy)-3-methylpyridine-4-carbonitrile;
2-{[(3R,6R)-1-{[5-fluoro-2-(2H-1,2,3-triazol-2-yl)phenyl]carbonyl}-6-methylpiperidin-3-yl]oxy}-3-methylpyridine-4-carbonitrile;
3-methyl-2-{[(3R,6R)-6-methyl-1-{[2-(2H-tetrazol-2-yl)phenyl]carbonyl}piperidin-3-yl]oxy}pyridine-4-carbonitrile;
2-{[(3R,6R)-1-{[5-cyano-2-(2H-1,2,3-triazol-2-yl)phenyl]carbonyl}-6-methylpiperidin-3-yl]oxy}-3-methylpyridine-4-carbonitrile;
2-{[(3R,6R)-1-{[6-chloro-2-(2H-1,2,3-triazol-2-yl)pyridin-3-yl]carbonyl}-6-methylpiperidin-3-yl]oxy}-3-methylpyridine-4-carbonitrile;
2-({(3R,6R)-1-[(2,6-dimethoxypyridin-3-yl)carbonyl]-6-methylpiperidin-3-yl}oxy)-3-methylpyridine-4-carbonitrile;
3-methyl-2-{[(3R,6R)-6-methyl-1-{[2-(pyrimidin-2-yl)phenyl]carbonyl}piperidin-3-yl]oxy}pyridine-4-carbonitrile;
3-methyl-2-({(3R,6R)-6-methyl-1-[(3-pyrimidin-2-ylthiophen-2-yl)carbonyl]piperidin-3-yl}oxy)pyridine-4-carbonitrile;
3-methyl-2-({(3R,6R)-6-methyl-1-[(4-methyl-2-yrimidin-2-ylphenyl)carbonyl]piperidin-3-yl}oxy)pyridine-4-carbonitrile;
3-methyl-2-({(3R,6R)-6-methyl-1-[(2-yrimidin-2-ylthiophen-3-yl)carbonyl]piperidin-3-yl}oxy)pyridine-4-carbonitrile;
2-({(3R,6R)-1-[(6-methoxy-2-phenylpyridin-3-yl)carbonyl]-6-methylpiperidin-3-yl}oxy)-3-methylpyridine-4-carbonitrile;
2-{[(3R,6R)-1-{[6-methoxy-2-(1-methyl-1H-pyrazol-5-yl)pyridin-3-yl]carbonyl}-6-methylpiperidin-3-yl]oxy}-3-methylpyridine-4-carbonitrile;
2-{[(3R,6R)-1-{[6-methoxy-2-(1-methyl-1H-pyrazol-4-yl)pyridin-3-yl]carbonyl}-6-methylpiperidin-3-yl]oxy}-3-methylpyridine-4-carbonitrile;
2-{[(3R,6R)-1-{[6-methoxy-2-(1-methyl-1H-pyrazol-3-yl)pyridin-3-yl]carbonyl}-6-methylpiperidin-3-yl]oxy}-3-methylpyridine-4-carbonitrile;
2-({(3R,6R)-1-[(6-methoxy-2,3′-bipyridin-3-yl)carbonyl]-6-methylpiperidin-3-yl}oxy)-3-methylpyridine-4-carbonitrile;
2-({(3R,6R)-1-[(6-methoxy-2,2′-bipyridin-3-yl)carbonyl]-6-methylpiperidin-3-yl}oxy)-3-methylpyridine-4-carbonitrile;
2-{[(3R,6R)-1-{[6-Methoxy-2-(methylsulfanyl)pyridin-3-yl]carbonyl}-6-methylpiperidin-3-yl]oxy}-3-methylpyridine-4-carbonitrile;
2-{[(3R,6R)-1-{[6-methoxy-4-(2H-1,2,3 riazol-2-yl)pyridin-3-yl]carbonyl}-6-methylpiperidin
3-yl]oxy}-3-methylpyridine-4-carbonitrile;
3-methyl-2-{[(3R,6R)-6-methyl-{[6-(methylsulfanyl)-2-(2H-1,2,3-triazol-2-yl)pyridin-3-yl]carbonyl}piperidin-3-yl]oxy}pyridine-4-carbonitrile;
2-{[(3R,6R)-1-{[2-(dimethylamino)-6-methoxypyridin-3-yl]carbonyl}-6-methylpiperidin-3-yl]oxy}-3-methylpyridine-4-carbonitrile;
2-{[(3R,6R)-1-{[6-(fluoromethoxy)-2-(2H-1,2,3-triazol-2-yl)pyridin-3-yl]carbonyl}-6-methylpiperidin-3-yl]oxy}-3-methylpyridine-4-carbonitrile;
2-{[(3R,6R)-1-{[6-bromo-2-(2H-1,2,3-triazol-2-yl)pyridin-3-yl]carbonyl}-6-methylpiperidin-3 yl]oxy}-3-methylpyridine-4-carbonitrile;
2-{[(3R,6R)-1-{[6-ethenyl-2-(2H-1,2,3 riazol-2-yl)pyridin-3-yl]carbonyl}-6-methylpiperidin-yl]oxy}-3-methylpyridine-4-carbonitrile;
3-chloro-2-{[(3R,6R)-6-methyl-1-{[2-(2H-1,2,3-triazol-2-yl)phenyl]carbonyl}piperidin-3-yl]oxy}pyridine-4-carbonitrile;
3-cyclopropyl-2-{[(3R,6R)-6-methyl-1-{[2-(2H-1,2,3-triazol-2-yl)phenyl]carbonyl}piperidin-3 yl]oxy}pyridine-4-carbonitrile;
3-ethyl-2-{[(3R,6R)-6-methyl-1-{[2-(2H-1,2,3-triazol-2-yl)phenyl]carbonyl}piperidin-3-yl]oxy}pyridine-4-carbonitrile;
2-{[(3R,6R)-6-methyl-1-{[2-(2H-1,2,3-triazol-2-yl)phenyl]carbonyl}piperidin-3-yl]oxy}pyridine-3,4-dicarbonitrile;
3-(methylsulfanyl)-2-{[(3R,6R)-6-methyl-1-{[2-(2H-1,2,3-triazol-2-yl)phenyl]carbonyl}piperidin-3-yl]oxy}pyridine-4-carbonitrile;
3-methoxy-2-{[(3R,6R)-6-methyl-1-{[2-(2H-1,2,3-triazol-2-yl)phenyl]carbonyl}piperidin-3-yl]oxy}pyridine-4-carbonitrile;
2-{[(3R,6R)-1-{[3-fluoro-2-(2H-1,2,3-triazol-2-yl)phenyl]carbonyl}-6-methylpiperidin-3-yl]oxy}-3-methoxypyridine-4-carbonitrile;
2-{[(3R,6R)-1-{[4-fluoro-2-(2H-1,2,3-triazol-2-yl)phenyl]carbonyl}-6-methylpiperidin-3-yl]oxy}-3-methoxypyridine-4-carbonitrile;
2-{[(3R,6R)-1-{[5-fluoro-2-(2H-1,2,3-triazol-2-yl)phenyl]carbonyl}-6-methylpiperidin-3-yl]oxy}-3-methoxypyridine-4-carbonitrile;
2-{[(3R,6R)-1-{[2-fluoro-6-(2H-1,2,3-triazol-2-yl)phenyl]carbonyl}-6-methylpiperidin-3-yl]oxy}-3-methoxypyridine-4-carbonitrile;
3-methoxy-2-{[(3R,6R)-6-methyl-1-{[3-methyl-2-(2H-1,2,3-triazol-2-yl)phenyl]carbonyl}piperidin-3-yl]oxy}pyridine-4-carbonitrile;
3-methoxy-2-{[(3R,6R)-6-methyl-1-{[4-methyl-2-(2H-1,2,3-triazol-2-yl)phenyl]carbonyl}piperidin-3-yl]oxy}pyridine-4-carbonitrile;
3-methoxy-2-{[(3R,6R)-6-methyl-1-{[5-methyl-2-(2H-1,2,3-triazol-2-yl)phenyl]carbonyl}piperidin-3-yl]oxy}pyridine-4-carbonitrile;
3-methoxy-2-{[(3R,6R)-1-{[6-methoxy-2-(2H-1,2,3-triazol-2-yl)pyridin-3-yl]carbonyl}-6-methylpiperidin-3-yl]oxy}pyridine-4-carbonitrile;
2-({(3R,6R)-1-[(2-cyclo butylphenyl)carbonyl]-6-methylpiperidin-3-yl}oxy)-3-methoxypyridine-4-carbonitrile;
3-methoxy-2-{[(3R,6R)-6-methyl-1-{[2-(2H-1,2,3-triazol-2-yl)thiophen-3-yl]carbonyl}piperidin-3-yl]oxy}pyridine-4-carbonitrile;
3-methoxy-2-({(3R,6R)-6-methyl-1-{[(2-pyrimidin-2-yl)phenyl]carbonyl}piperidin-3-yl}oxy)pyridine-4-carbonitrile;
3-methoxy-2-({(3R,6R)-6-methyl-1-[(2-pyrimidin-2-ylthiophen-3-yl)carbonyl]piperidin-3-yl}oxy)pyridine-4-carbonitrile;
methyl 4-cyano-2-{[(3R,6R)-1-{[3-fluoro-2-(pyrimidin-2-yl)phenyl]carbonyl}-6-methylpiperidin-3-yl]oxy}pyridine-3-carboxylate
2-({(3R,6R)-1-[(4-fluoro-2̂yrimidin-2-ylphenyl)carbonyl]-6-methylpiperidin-3-yl}oxy)-3-methoxypyridine-4-carbonitrile;
2-({(3R,6R)-1-[(5-fluoro-2̂yrimidin-2-ylphenyl)carbonyl]-6-methylpiperidin-3-yl}oxy)-3-methoxypyridine-4-carbonitrile;
2-({(3R,6R)-1-[(2-fluoro-6̂yrimidin-2-ylphenyl)carbonyl]-6-methylpiperidin-3-yl}oxy)-3-methoxypyridine-4-carbonitrile;
3-methoxy-2-({(3R,6R)-1-[(6-methoxy-2-pyrimidin-2-ylpyridin-3-yl)carbonyl]-6-methylpiperidin-3-yl}oxy)pyridine-4-carbonitrile;
3-methyl-2-{[(3R,6R)-6-methyl-1-{[2-(1-methylethoxy)pyridin-3-yl]carbonyl}piperidin-3-yl]oxy}pyridine-4-carbonitrile; and
3-methoxy-2-{[(3R,6R)-6-methyl-1-{[2-(2H-tetrazol-2-yl)phenyl]carbonyl}piperidin-3-yl]oxy}pyridine-4-carbonitrile;
and pharmaceutically acceptable salts thereof.

In some embodiments, the OX1R antagonist of the present invention is selected from 2-pyridylamino-4-nitrile-piperidinyl orexin receptor antagonists that are disclosed in WO 2014085208 A1. In some embodiments, the OX1R antagonist of the present invention is selected from the group consisting of:
2-(43R,6R)-1-(2-(2H-1,2,3-triazol-2-yl)benzoyl)-6-methylpiperidin-3-yl)amino)isonicotinonitrile;
3-methoxy-2-{[(3R,6R)-6-methyl-1-{[2-(2H-1,2,3-triazol-2-yl)phenyl]carbonyl}piperidin-3-yl]amino}pyridine-4-carbonitrile;
2-(((3R,6R)-1-(2-(2H-tetrazol-2-yl)benzoyl)-6-methylpiperidin-3-yl)amino)isonicotinonitrile;
2-(((3R,6R)-1-(2-(2H-1,2,3-triazol-2-yl)benzoyl)-6-methylpiperidin-3-yl)amino)-3-methylisonicotinonitrile;
3-methyl-2-{[(3R,6R)-6-methyl-1-{[2-(2H-tetrazol-2-yl)phenyl]carbonyl}piperidin-3-yl]amino}pyridine-4-carbonitrile;
2-((3R,6R)-1-(2-(2H-1,2,3-triazol-2-yl)thiophene-3-carbonyl)-6-methylpiperidin-3-ylamino)-3 methoxyisonicotinonitrile;
3-methoxy-2-((3R,6R)-6-methyl-1-(24 yrimidin-2-yl)thiophene-3-carbonyl)piperidin-3-ylamino)isonicotinonitrile;
2-(43R,6R)-1-(2-(2H-1,2,3-triazol-2-yl)benzoyl)-6-methylpiperidin-3-yl)(methyl)amino)isonicotinonitrile;
2-{methyl[(3R,6R)-6-methyl-1-{[2-(2H-tetrazol-2-yl)phenyl]carbonyl}piperidin-3-yl]amino}pyridine-4-carbonitrile;
3-methoxy-2-{methyl[(3R,6R)-6-methyl-1-{[2-(2H-1,2,3-triazol-2-yl)thiophen-3-yl]carbonyl}piperidin-3-yl]amino}pyridine-4-carbonitrile;
3-methoxy-2-(methyl {(3R,6R)-6-methyl-1-[(2-pyrimidin-2-ylthiophen-3-yl)carbonyl]piperidin 3-yl}amino)pyridine-4-carbonitrile;
2-{ethyl[(3R,6R)-6-methyl-1-{[2-(2H-1,2,3-triazol-2-yl)phenyl]carbonyl}piperidin-3-yl]amino}pyridine-4-carbonitrile;
2-{[(3R,6R)-6-methyl-1-{[2-(2H-1,2,3-triazol-2-yl)phenyl]carbonyl}piperidin-3-yl](prop-2-en-1-yl)amino}pyridine-4-carbonitrile;
2-{[(3R,6R)-6-methyl-1-{[2-(2H-1,2,3-triazol-2-yl)phenyl]carbonyl}piperidin-3-yl] (propyl)amino}pyridine-4-carbonitrile;
N-((3R,6R)-1-(2-(2H-1,2,3-triazol-2-yl)benzoyl)-6-methylpiperidin-3-yl)-N-(4-cyanopyridin-2 yl)acetamide;
methyl 4-cyano-2-{[(3R,6R)-6-methyl-1-{[2-(2H-1,2,3-triazol-2-yl)phenyl]carbonyl}piperidine-3-yl]amino}pyridine-3-carboxylate;
methyl 4-cyano-2-({(3R,6R)-6-methyl-1-[(2-pyrimidin-2-ylphenyl)carbonyl]piperidin-3-yl}amino)pyridine-3-carboxylate;
and pharmaceutically acceptable salts thereof.

In some embodiments, the OX1R antagonist of the present invention is selected from 2-pyridyloxy-4-nitrile orexin receptor antagonists that are disclosed in WO 2013059222 A1. In some embodiments, the OX1R antagonist of the present invention is selected from the group consisting of
2-{[(3R,6R)-6-methyl-1-{[2-(2H-1,2,3-triazol-2-yl)phenyl]carbonyl}piperidin-3-yl]oxy}pyridine-4-carbonitrile;
2-({(3R,6R)-6-methyl-1-[(2-pyrimidin-2-ylphenyl)carbonyl]piperidin-3-yl}oxy)pyridine-4-carbonitrile;
2-{[(3R,6R)-6-methyl-1-{[2-(5-methyl-1,2,4-oxadiazol-3-yl)phenyl]carbonyl}piperidin-3-yl]oxy}pyridine-4-carbonitrile;
2-{[(3R,6R)-6-methyl-1-{[2-methyl-6-(2H-1,2,3-triazol-2-yl)phenyl]carbonyl}piperidin-3-yl]oxy}pyridine-4-carbonitrile;
2-{[(3R,6R)-6-methyl-1-[3-methyl-2-(2H-1,2,3-triazol-2-yl)phenyl]carbonyl piperidin-3-yl]oxy}pyridine-4-carbonitrile;
2-{[(3R,6R)-1-{[3-fluoro-2-(2H-1,2,3-triazol-2-yl)phenyl]carbonyl}-6-methylpiperidin-3-yl]oxy}pyridine-4-carbonitrile;
2-{[(3R,6R)-1-{[4-chloro-2-(2H-1,2,3-triazol-2-yl)phenyl]carbonyl}-6-methylpiperidin-3-yl]oxy}pyridine-4-carbonitrile;
2-{[(3R,6R)-1-{[4-fluoro-2-(2H-1,2,3-triazol-2-yl)phenyl]carbonyl}-6-methylpiperidin-3-yl]oxy}pyridine-4-carbonitrile;
2-{[(3R,6R)-6-methyl-1-{[4-methyl-2-(2H-1,2,3-triazol-2-yl)phenyl]carbonyl}piperidin-3-yl]oxy}pyridine-4-carbonitrile;
2-{[(3R,6R)-6-methyl-1-{[2-(2H-1,2,3-triazol-2-yl)thiophen-3-yl]carbonyl}piperidin-3-yl]oxy}pyridine-4-carbonitrile;
2-{[(3R,6R)-6-methyl-1-{[4-(2H-1,2,3-triazol-2-yl)isothiazol-3-yl]carbonyl}piperidin-3-yl]oxy}pyridine-4-carbonitrile;
2-{[(3R,6R)-6-methyl-1-{[1-methyl-3-(2H-1,2,3-triazol-2-yl)-1H-pyrazol-4-yl]carbonyl}piperidin-3-yl]oxy}pyridine-4-carbonitrile;
2-{[(3R,6R)-6-methyl-1-{[3-methyl-5-(2H-1,2,3-triazol-2-yl)isothiazol-4-yl]carbonyl}piperidin-3-yl]oxy}pyridine-4-carbonitrile;
2-{[(3R,6R)-6-methyl-1-{[3-(2H-1,2,3-triazol-2-yl)thiophen-2-yl]carbonyl}piperidin-3-yl]oxy}pyridine-4-carbonitrile;
2-{[(3R,6R)-1-{[5-bromo-2-(2H-1,2,3-triazol-2-yl)phenyl]carbonyl}-6-methylpiperidin-3-yl]oxy}pyridine-4-carbonitrile;
2-{[(3R,6R)-1-{[5-chloro-2-(2H-1,2,3-triazol-2-yl)phenyl]carbonyl}-6-methylpiperidin-3-yl]oxy}pyridine-4-carbonitrile;
2-{[(3R,6R)-1-{[6-methoxy-2-(2H-1,2,3-triazol-2-yl)pyridin-3-yl]carbonyl}-6-methylpiperidin-3-yl]oxy}pyridine-4-carbonitrile;
3-({(2R,5R)-5-[(4-cyanopyridin-2-yl)oxy]-2-methylpiperidin-1-yl}carbonyl)-4-(2H-1,2,3-triazol-2-yl)benzamide;
2-{[(3R,6R)-1-{[4-cyano-2-(2H-1,2,3-triazol-2-yl)phenyl]carbonyl}-6-methylpiperidin-3-yl]oxy}pyridine-4-carbonitrile;
2-({(3R,6R)-1-[(2-cyclopropyl-6-methoxypyridin-3-yl)carbonyl]-6-methylpiperidin-3-yl}oxy)pyridine-4-carbonitrile;
2-{[(3R,6R)-1-{[4-ethoxy-2-(2H-1,2,3-triazol-2-yl)phenyl]carbonyl}-6-methylpiperidin-3-yl]oxy}pyridine-4-carbonitrile;
2-{[(3R,6R)-1-{[2-fluoro-6-(2H-1,2,3-triazol-2-yl)phenyl]carbonyl}-6-methylpiperidin-3-yl]oxy}pyridine-4-carbonitrile;
2-{[(3R,6R)-1-{[4-(fluoromethoxy)-2-(2H-1,2,3-triazol-2-yl)phenyl]carbonyl}-6-methylpiperidin-3-yl]oxy}pyridine-4-carbonitrile;
2-{[(3R,6R)-1-{[4-(difluoromethoxy)-2-(2H-1,2,3-triazol-2-yl)phenyl]carbonyl}-6-methylpiperidin-3-yl]oxy}pyridine-4-carbonitrile;
2-{[(3R,6R)-1-{[4-(2-hydroxyethoxy)-2-(2H-1,2,3-triazol-2-yl)phenyl]carbonyl}-6-methylpiperidin-3-yl]oxy}pyridine-4-carbonitrile;
2-{[(3R,6R)-6-methyl-1-{[6-methyl-2-(2H-1,2,3-triazol-2-yl)pyridin-3-yl]carbonyl}piperidin-3 yl]oxy}pyridine-4-carbonitrile;
2-{[(3R,6R)-6-methyl-1-{[2-(2H-1,2,3-triazol-2-yl)-6-(trifluoromethyl)pyridin-3-yl]carbonyl}piperidin-3-yl]oxy}pyridine-4-carbonitrile;
2-({3R,6R)-1-[(2-chloro-6-methoxypyridin-3-yl)carbonyl]-6-methylpiperidin-3-yl}oxy)pyridine-4-carbonitrile;
2-{[(3R,6R)-1-{[4-chloro-2-(2H-1,2,3-triazol-2-yl)phenyl]carbonyl}-6-methylpiperidin-3-yl]oxy}pyridine-4-carbonitrile;
2-{[(3R,6R)-6-methyl-1-[2-(2H-tetrazol-2-yl)phenyl]carbonyl piperidin-3-yl]oxy}pyridine-4-carbonitrile;
2-({(3R,6R)-6-methyl-1-[(3-pyrimidin-2-ylthiophen-2-yl)carbonyl]piperidin-3-yl}oxy)pyridine-4-carbonitrile;
2-({(3R,6R)-6-methyl-1-[(2-pyrimidin-2-ylthiophen-3-yl)carbonyl]piperidin-3-yl}oxy)pyridine-4-carbonitrile;
2-({(3R,6R)-1-[(3-fluoro-2-pyrimidin-2-ylphenyl)carbonyl]-6-methylpiperidin-3-yl}oxy)pyridine-4-carbonitrile;
2-({(3R,6R)-1-[(4-fluoro-2-pyrimidin-2-ylphenyl)carbonyl]-6-methylpiperidin-3-yl}oxy)pyridine-4-carbonitrile;
2-({(3R,6R)-1-[(5-fluoro-2-pyrimidin-2-ylphenyl)carbonyl]-6-methylpiperidin-3-yl}oxy)pyridine-4-carbonitrile; and
2-({(3R,6R)-1-[(2-fluoro-6-pyrimidin-2-ylphenyl)carbonyl]-6-methylpiperidin-3-yl}oxy)pyridine-4-carbonitrile;
and pharmaceutically acceptable salts thereof.

In some embodiments, the OX1R antagonist of the present invention is selected from 2-pyridyloxy-4-ester orexin receptor antagonists that are disclosed in WO 2014099696 A1. In some embodiments, the OX1R antagonist of the present invention is selected from the group consisting of:
methyl 2-{[(3R,6R)-6-methyl-1-{[2-(2H-1,2,3-triazol-2-yl)phenyl]carbonyl}piperidin-3-yl]oxy}pyridine-4-carboxylate;
2-{[(3R,6R)-6-methyl-1-{[2-(2H-1,2,3-triazol-2-yl)phenyl]carbonyl}piperidin-3-yl]oxy}pyridine-4-carboxylic acid;
methyl 2-{[(3R,6R)-6-methyl-1-{[2-(2H-1,2,3-triazol-2-yl)phenyl]carbonyl}piperidin-3-yl]oxy}-5-(trifluoromethyl)pyridine-4-carboxylate;
methyl 5-bromo-2-{[(3R,6R)-6-methyl-1-{[2-(2H-1,2,3-triazol-2-yl)phenyl]carbonyl}piperidin-3-yl]oxy}pyridine-4-carboxylate;
methyl 3-chloro-2-{[(3R,6R)-6-methyl-1-{[2-(2H-1,2,3-triazol-2-yl)phenyl]carbonyl}piperidin-3-yl]oxy}pyridine-4-carboxylate;
dimethyl 2-{[(3R,6R)-6-methyl-1-{[2-(2H-1,2,3-triazol-2-yl)phenyl]carbonyl}piperidin-3-yl]oxy}pyridine-3,4-dicarboxylate;
methyl 2-methyl-6-{[(3R,6R)-6-methyl-1-{[2-(2H-1,2,3-triazol-2-yl)phenyl]carbonyl}piperidin-3-yl]oxy}pyridine-4-carboxylate;
methyl 3-fluoro-2-{[(3R,6R)-6-methyl-1-{[2-(2H-1,2,3-triazol-2-yl)phenyl]carbonyl}piperidin-3-yl]oxy}pyridine-4-carboxylate;
methyl 2-{[(3R,6R)-1-{[6-methoxy-2-(2H-1,2,3-triazol-2-yl)pyridin-3-yl]carbonyl}-6-methylpiperidin-3-yl]oxy}pyridine-4-carboxylate;
methyl 5-methoxy-2-{[(3R,6R)-6-methyl-1-{[2-(2H-1,2,3-triazol-2-yl)phenyl]carbonyl}piperidin-3-yl]oxy}pyridine-4-carboxylate;
methyl 5-methyl-2-{[(3R,6R)-6-methyl-1-{[2-(2H-1,2,3-triazol-2-yl)phenyl]carbonyl}piperidin-3-yl]oxy}pyridine-4-carboxylate;
methyl 2-({(3R,6R)-1-[(6-methoxy-2-pyrimidin-2-ylpyridin-3-yl)carbonyl]-6-methylpiperidin-3-yl}oxy)pyridine-4-carboxylate;
and pharmaceutically acceptable salts thereof.

In some embodiments, the OX1R antagonist of the present invention is selected from tertiary amide orexin receptor antagonists that are disclosed in WO 2011053522 A1. In some embodiments, the OX1R antagonist of the present invention are selected from the group consisting of:
N-[2-(5,6-dimethoxy-3 pyridinyl)ethyl]-N-[3-(4-methoxyphenyl)-1-methylpropyl]-6-methyl-2-pyridinecarboxamide;
6-chloro-N-[2-(5,6-dimethoxypyridinecarboxamide;
N-f2-(5,6-dimethoxy-2-pyridinyl)ethyi]-6-methyl-N-(1-methyl-3-phenylpropyl)-2-pyridinecarboxamide;
N-[2-(5,6-dimethoxypyridin-2-yl)ethyl]-6-(dimethylamino)-N-[4-(4-methoxyphenyl)butan-2-yl]pyridine-2-carboxamide;
6-chloro-N-[2-(5,6-dimethoxypyridin-2-yl)ethyl]-N-(4-phenylbutan-2-yl)pyridine-2-carboxamide
N-[2-(5_{}}6-dimethoxypyridin-2-yI)ethyl]-3-methyl-N-(4-phenylbutan-2-yl)benzamide;
6-bromo-N-[2-(5,6-dimethoxypyridin-2-yl)ethyl]-N-(4-phenylbutan-2-yl)pyridine-2-carboxamide;
N-[2-(5,6-dimethoxypyridin-2-yl)ethyl]-6-fluoro-N-(4-phenylbutan-2-yl)pyridine-2-carboxamide;
N-[2-(5,6-dimethoxypyridin-2-yl)ethyl]-N-(4-phenylbutan-2-yl)-6-(propan-2-yl)pyridine-2-carboxamide;
6-cyano-N-[2-(5,6-di methoxypyridin-2-yl)ethyl]-N-(4-phenylbutan-2-yl)pyridine-2-carboxamide
6-cyclopropyl-N-[2-(5,6-dimethoxypyridin-2-yl)ethyl]-N-(4-phenylbutan-2-yl)pyridine-2-carboxamide;
N-[2-(5,6-dimethoxypyridin-2-yl)ethyl]-6-ethyl-N-(4-phenylbutan-2-yl)pyridine-2-carboxamide
N-[2-(5,6-dimethoxypyridin-2-yl)ethyl]-6-methylcarboxamide;
6-chloro-N-[2-(5,6-dimethoxypyridin-2-yl)methylcarboxamide;
and pharmaceutically acceptable salts thereof,

In some embodiments, the OX1R antagonist of the present invention is selected from 3-ester-4-substituted orexin receptor antagonists that are disclosed in WO 2014099697 A1. In some embodiments, the OX1R antagonist of the present invention is selected from the group consisting of:
ethyl 4-methyl-2-{[(3R,6R)-6-methyl-1-{[2-(2H-1,2,3-triazol-2-yl)phenyl]carbonyl}piperidin-3-yl]oxy}pyridine-3-carboxylate;
methyl 4-methyl-2-{[(3R,6R)-6-methyl-1-{[2-(2H-1,2,3-triazol-2-yl)phenyl]carbonyl}piperidin 3-yl]oxy}pyridine-3-carboxylate;
methyl 4-(methylsulfanyl)-2-{[(3R,6R)-6-methyl-1-{[2-(2H-1,2,3-triazol-2-yl)phenyl]carbonyl}piperidin-3-yl]oxy}pyridine-3-carboxylate;
methyl 2-({(3R,6R)-6-methyl-1-[(2-pyrimidin-2-ylphenyl)carbonyl]piperidin-3-yl}oxy)-4-(methylsulfanyl)pyridine-3-carboxylate;
methyl 2-({(3R,6R)-1-[(5-fluoro-2-pyrimidin-2-ylphenyl)carbonyl]-6-methylpiperidin-3-yl}oxy)-4-(methylsulfanyl)pyridine-3-carboxylate;
methyl 2-({(3R,6R)-1-[(4-fluoro-2-pyrimidin-2-ylphenyl)carbonyl]-6-methylpiperidin-3-yl}oxy)-4-(methylsulfanyl)pyridine-3-carboxylate;
methyl 2-({(3R,6R)-1-[(3-fluoro-2-pyrimidin-2-ylphenyl)carbonyl]-6-methylpiperidin-3-yl}oxy)-4-(methylsulfanyl)pyridine-3-carboxylate;
methyl 4-azetidin-1-yl-2-{[(3R,6R)-6-methyl-1-{[2-(2H-1,2,3-triazol-2-yl)phenyl]carbonyl}piperidin-3-yl]oxy}pyridine-3-carboxylate;
methyl 4-(4-methylpiperazin-1-yl)-2-{[(3R,6R)-6-methyl-1-{[2-(2H-1,2,3-triazol-2-yl)phenyl]carbonyl}-piperidin-3-yl]oxy}pyridine-3-carboxylate;
methyl 4-ethyl-2-{[(3R,6R)-6-methyl-1-{[2-(2H-1,2,3-triazol-2-yl)phenyl]carbonyl}piperidin-3 yl]oxy}pyridine-3-carboxylate;
methyl 4-tert-butyl-2-{[(3R,6R)-6-methyl-1-{[2-(2H-1,2,3-triazol-2-yl)phenyl]carbonyl}piperidin-3-yl]oxy}pyridine-3-carboxylate;
methyl 4-(1-methylethyl)-2-{[(3R,6R)-6-methyl-1-{[2-(2H-1,2,3-triazol-2-yl)phenyl]carbonyl}piperidin-3-yl]oxy}pyridine-3-carboxylate;
methyl 4-cyclopropyl-2-{[(3R,6R)-6-methyl-1-{[2-(2H-1,2,3-triazol-2-yl)phenyl]carbonyl}piperidin-3-yl]oxy}pyridine-3-carboxylate;
methyl 4-cyclobutyl-2-{[(3R,6R)-6-methyl-1-{[2-(2H-1,2,3-triazol-2-yl)phenyl]carbonyl}piperidin-3-yl]oxy}pyridine-3-carboxylate;
methyl 2-methyl-6-{[(3R,6R)-6-methyl-1-{[2-(2H-1,2,3-triazol-2-yl)phenyl]carbonyl}piperidin 3-yl]oxy}benzoate;
methyl 2-(1-methylethyl)-6-{[(3R,6R)-6-methyl-1-{[2-(2H-1,2,3-triazol-2-yl)phenyl]carbonyl}piperidin-3-yl]oxy}benzoate; and
ethyl 4-ethyl-2-{[(3R,6R)-6-methyl-1-{[2-(2H-1,2,3-triazol-2-yl)phenyl]carbonyl}piperidin-3-yl]oxy}pyridine-3-carboxylate;
and pharmaceutically acceptable salts thereof.

In some embodiments, the OX1R antagonist of the present invention is selected from the group consisting of 2,5-disubstituted thiomorpholine orexin receptor antagonists that are disclosed in WO 2013059163 A1. In some embodiments, the OX1R antagonist of the present invention is selected from the group consisting of:
[(2R,5R)-2-{[(5-fluoropyridin-2-yl)oxy]methyl}-5-methylthiomorpholin-4-yl][5-methyl-2-(2H-1,2,3-triazol-2-yl)phenyl]methanone;
(2R,5R)-5-methyl-2-[(pyridin-2-yloxy)methyl]-4-{[2-(2H-1,2,3-triazol-2-yl)phenyl]carbonyl}thiomorpholine;
(2R,5R)-2-{[(5-fluoropyridin-2-yl)oxy]methyl}-5-methyl-4-{[2-(2H-1,2,3-triazol-2-yl)phenyl]carbonyl}thiomorpholine;
(2R,5R)-5-methyl-2-{[(5-methylpyridin-2-yl)oxy]methyl}-4-{[2-(2H-1,2,3-triazol-2-yl)phenyl]carbonyl}thiomorpholine;
(2R,5R)-2-{[(5-chloropyridin-2-yl)oxy] methyl}-5-methyl-4-{[2-(2H-1,2,3-triazol-2-yl)phenyl]carbonyl}thiomorpholine;
6-{[(2R,5R)-5-methyl-4-{[2-(2H-1,2,3-triazol-2-yl)phenyl]carbonyl}thiomorpholin-2-yljmethoxyl}pyridine-3-carbonitrile;
(2R,5R)-5-methyl-4-{[2-(2H-1,2,3-triazol-2-yl)phenyl]carbonyl}-2-({[5-(trifluoromethyl)pyridin-2-yl]oxy}methyl)thiomorpholine;
(2R,5R)-2-{[(3-chloropyridin-2-yl)oxy]methyl}-5-methyl-4-{[2-(2H-1,2,3-triazol-2-yl)phenyl]carbonyl}thiomorpholine;
2-{[(2R,5R)-5-methyl-4-{[2-(2H-1,2,3-triazol-2-yl)phenyl]carbonyl}thiomorpholin-2-yljmethoxy}pyridine-3-carbonitrile;
(2R,5R)-5-methyl-2-{[(4-methylpyridin-2-yl)oxy]methyl}-4-{[2-(2H-1,2,3-triazol-2-yl)phenyl]carbonyl}thiomorpholine;
(2R,5R)-5-methyl-2-[(pyrimidin-2-yloxy)methyl]-4-{[2-(2H-1,2,3-triazol-2-yl)phenyl]carbonyl}thiomorpholine;
(2R,5R)-2-{[(5-chloro-4-methylpyrimidin-2-yl)oxy]methyl}-5-methyl-4-{[2-(2H-1,2,3-triazol-2-yl)phenyl]carbonyl}thiomorpholine;
(2R,5R)-2-{[(4-chloro-5-methylpyrimidin-2-yl)oxy]methyl}-5-methyl-4-{[2-(2H,2,3-M yl)phenyl]carbonyl}thiomorpholine;
(2R,5R)-2-{[(4-chloro-5-methoxypyrimidin-2-yl)oxy]methyl}-5-methyl-4-{[2-(2H-1,22-yl)phenyl]carbonyl}thiomorpholine;
[(2R,5R)-2-{[(5-fluoropyridin-2-yl)oxy]methyl}-5-methyl-1-oxidothiomorpholin-4-1,2,3-triazol-2-yl)phenyl]methanone;
[(2R,5R)-2-{[(5-fluoropyridin-2-yl)oxy]methyl}-5-m(2H-1,2,3-triazol-2-yl)phenyl]methanone; and
{(2R,5R)-5-methyl-2-[(pyridine-2-ylsulfanyl)methyl]thiomorpholin-4-yl} [2-(2H-1,2,3-triazol-2-yl)phenyl]methanone;
and pharmaceutically acceptable salt thereof.

In some embodiments, the OX1R antagonist of the present invention is selected from piperidinyl alkyne orexin receptor antagonists that are disclosed in WO 2013062857 A1. In some embodiments, the OX1R antagonist of the present invention is selected from the group consisting of:
[(2R,5R)-2-memyl-5-(yridin-2-ylemynyl)piperidin-1-yl][2-(2H-1,2,3-triyl)phenyl]methanone;
2-{[(3S,6R)-6-methyl-1-{[2-(2H-1,2,3-triazol-2-yl)phenyl]carbonyl}piperidin-3-yl] ethynyl} pyridine;
4-{[(3S,6R)-6-memyl-1-{[2-(2H,2₅3-tria2ol-2-yl)phenyl]carbonyl}piperidin-3-yl] ethynyl} pyridine;
3-{[(3 S,6R)-6-methyl-1-{[2-(2H-1,2,3-triazol-2-yl)phenyl]carbonyl}piperidin-3-yl] ethynyl} pyridine;
3-{[(3 S,6R)-6-methyl-1-{[2-(2H-1,2,3-triazol-2-yl)phenyl]carbonyl}piperidin-3-yl] ethynyl} pyridin-2-ol;
3-{[(3S,6R)-6-methyl-1-{[2-(2H-1,2_{}}3-triazol-2-yl)phenyl]carbonyl}piperidin-3-yl]ethynyl}pyridin-4-ol;
(5-{[(3S,6R)-6-merayl-1-{[2-(2H-1,2,3-triayl]ethynyl}pyridin-2-yl)methanol;
(6-{[(3 S,6R)-6-methyl-1-{[2-(2H-1,2,3-triazol-2-yl)phenyl]carbonyl}piperidin-3-yl] ethynyl} pyridin-3-yl)methanol;
(6-{[(3S,6R)-6-methyl-1-{[2-(2H-1,2,3-triazol-2-yl)phenyl]carbonyl}piperidin-3-yl] ethynyl} pyridin-2-yl)methanol;
(2-{[(3S,6R)-6-memyl-1-{[2-(2H,2,3-triazol-2-yl)phenyl]carbonyl}piperidin-3-yl]ethynyl}pyridin-4-yl)methanol;
(4-{[(3S,6R)-6-methyl-1-{[2-(2H-1,2_{J}3-triazol-2-yl)phenyl]carbonyl}piperidin-4-yl] ethynyl} pyridin-2-yl)methanol;
{(2R.5S)-5-[(5-fluoropyridin-2-yl)ethynyl]-2-methylpiperidin-1-yl} [5-methyl-2-(2H-1,2,3-triazol-2-yl)phenyl]methanone;
[5-methyl-2-(2H-1,2,3-azol-2-yl)phenyl][2-(phenylethynyl)piperidin-1-yl 1-{[5-methyl-2-(2H-1,2,3-triazol-2-yl)phenyl]carbonyl}-2-(phenylethynyl)piperidine;
5-fluoro-2-[(1-{[5-methyl-2-(2H-1,2,3-triazol-2-yl)phenyl]carbonyl}piperidin-2-yl)ethynyl]pyridine;
2-[(4-fluorophenyl)ethynyl]-1-{[5-methyl-2-(2H-1,2,3-1xiazol-2-yl)phenyl] 2-[(1-{[5-methyl-2-(2H-1_{J}2,3-triazol-2-yl)phenyl]carbonyl}piperidin-2-yl)ethynyl]quinoline;
1-{[5-methyl-2-(2H-1,2,3-triazol-2-yl)phenyl]carbonyl}-2-(naphthalen-2-ylethynyl)piperidine;
(2-merayl-5-phenyl-1,3-thiazol-4-yl) [2-(phenylethynyl)piperidin-1-yl]methanone;
1-[(2-methyl-5-phenyl-1,3-thiazol-4-yl)carbonyl]-2-(phenylethynyl)piperidine;
and pharmaceutically acceptable salts thereof.

In some embodiments, the OX1R antagonist of the present invention is selected from 2-pyridyloxy-3-nitrile-4-substituted orexin receptor antagonists that are disclosed in WO 2014099698 A1. In some embodiments, the OX1R antagonist of the present invention is selected from the group consisting of:
4-(Methylsulfanyl)-2-{[(3R,6R)-6-methyl-1-{[2-(2H-1,2,3-triazol-2-yl)phenyl]carbonyl}-piperidin-3-yl]oxy}pyridine-3-carbonitrile;
4-Methyl-2-{[(3R,6R)-6-methyl-1-{[2-(2H-1,2,3-triazol-2-yl)phenyl]carbonyl}piperidin-3-yl]oxy}pyridine-3-carbonitrile;
2-{[(3R,6R)-1-{[4-Chloro-2-(2H-1,2,3-triazol-2-yl)phenyl]carbonyl}-6-methylpiperidin-3-yl]oxy}-4-methylpyridine-3-carbonitrile;
4-methyl-2-{[(3R,6R)-6-methyl-1-{[4-(2H-1,2,3-triazol-2-yl)pyridin-3-yl]carbonyl}piperidin-3 yl]oxy}pyridine-3-carbonitrile;
2-{[(3R,6R)-1-{[2-fluoro-6-(2H-1,2,3-triazol-2-yl)phenyl]carbonyl}-6-methylpiperidin-3-yl]oxy}-4-methylpyridine-3-carbonitrile;
2-{[(3R,6R)-1-{[2-methoxy-6-(2H-1,2,3-triazol-2-yl)phenyl]carbonyl}-6-methylpiperidin-3-yl]oxy}-4-methylpyridine-3-carbonitrile;
4-methyl-2-{[(3R,6R)-6-methyl-1-{[3-methyl-2-(2H-1,2,3-triazol-2-yl)phenyl]carbonyl}piperidin-3-yl]oxy}pyridine-3-carbonitrile;
2-{[(3R,6R)-1-{[4-fluoro-2-(2H-1,2,3-triazol-2-yl)phenyl]carbonyl}-6-methylpiperidin-3-yl]oxy}-4-methylpyridine-3-carbonitrile;
2-{[(3R,6R)-1-{[4-methoxy-2-(2H-1,2,3-triazol-2-yl)phenyl]carbonyl}-6-methylpiperidin-3-yl]oxy}-4-methylpyridine-3-carbonitrile;
2-{[(3R,6R)-1-{[3-fluoro-2-(2H-1,2,3-triazol-2-yl)phenyl]carbonyl}-6-methylpiperidin-3-yl]oxy}-4-methylpyridine-3-carbonitrile;
4-methyl-2-{[(3R,6R)-6-methyl-1-{[5-methyl-2-(2H-1,2,3-triazol-2-yl)phenyl]carbonyl}piperidin-3-yl]oxy}pyridine-3-carbonitrile;
2-{[(3R,6R)-1-{([6-methoxy-2-(2H-1,2,3-triazol-2-yl)pyridin-3-yl]carbonyl}-6-methylpiperidin-3-yl] oxy}-4-methylpyridine-3-carbonitrile;
and pharmaceutically acceptable salts thereof.

The terms “administer” or “administration” refer to the act of injecting or otherwise physically delivering a substance as it exists outside the body (e.g., a OX1R antagonist of the present invention) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In some embodiments, the OX1R antagonist of the invention is administered to the subject with a therapeutically effective amount.

By a “therapeutically effective amount” is meant a sufficient amount of OX1R to treat the inflammatory bowel disease at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In some embodiments, the OX1R antagonist of the present invention is administered to the subject in combination with a standard treatment. Typically the standard treatment is selected from the group consisting of corticosteroids, immunosuppressive drugs, aminosalicylates sulfasalazine, such as Mesalazine (also known as 5-aminosalicylic acid, mesalamine, or 5-ASA. Brand name formulations include Apriso, Asacol, Pentasa, Mezavant, Lialda, Fivasa, Rovasa and Salofalk.), Sulfasalazine (also known as Azulfidine), Balsalazide (also known as Colazal or Colazide (UK)), Olsalazine (also known as Dipentum), immunosuppressors (azathioprine, 6-mercaptopurine, methotrexate, rapamycine, cyclosporine and tacrolimus) or biological treatments such as Infliximab, Visilizumab, Adalimumab, or Vedolizumab, golimumab, tofacitinib.

The OX1R antagonist of the invention is typically combined with pharmaceutically acceptable excipients, and optionally sustained-release matrices, such as biodegradable polymers, to be administered in the form of a pharmaceutical composition. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The antibody can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active antibody in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1: OX1R is highly expressed in inflammatory areas of IBD patients but not in the normal colonic mucosa. Left, immunodetection of OX1R in normal colonic mucosa; Middle, immunodetection of OX1R in Crohn's disease (21 samples); Right, immunodetection of OX1R in ulcerative colitis (20 samples).

FIG. 2: OXA ameliorates the DAI (weight and colitis score) of DSS-induced colitis mice. Mice were orally treated with 5% DSS for 7 days in the presence or in the absence of daily intraperitoneal injection of OxA (0.22 μmoles/kg). Left, determination of gain or loss of weight in the absence of DSS treatment (Control, ▴), in the presence of DSS treatment (DSS, ▪) and in the presence of DSS associated to OxA treatment (DSS+OxA, ●). Right, evaluation of Disease Activity Index (DAI) scored by measuring weight, length of colon, diarrhea and blood presence in the stool in DSS untreated control mice (Control, ▴), in DSS treated mice (DSS, ▪) and in DSS treated mice associated to OxA treatment (DSS+OxA, ●).

FIG. 3: OXA effects on cytokines secretion in DSS-induced colitis mice. Right, Colons of DSS untreated mice (white), DSS treated mice (gray) and DSS treated mice associated to OxA treatment (black) were resected after animal sacrifice. Then, protein extraction was performed by tissue disruption. Cytokines were determined using Cytokine CBA kits (see Material and Methods). Left, after colon resection, RNA were extracted from colonic tissue and qPCR was performed using specific oligonucleotides for IL8 homolog and IL1B.

FIG. 4: Histological aspect of colon mucosa in OxA treated EXCY2 mice (right) and non-treated EXCY2 mice (left). EXCY2 mice genetically invalidated for IL10 and Nox1 genes (IL10^{-/-} and Nox1^{-/-}) which develop spontaneous UC-like disease, were treated with OxA (0.22 μmoles/kg, 2 intraperitoneal injection/week for 21 days) (right) or untreated (left). After animal sacrifice colons were resected and histologic analysis was performed.

FIG. 5: Effect of orexin-A and SB408124 antagonist on Ca2+ mobilization in HEK-OX1R cells. Top, HEK-OX1R cells were incubated with fluorescence probe (FluoForte) for 45 min. at 37° C. according to FluoForte calcium assay kit (Enzo Life Sciences). 1 μM of OxA was added to cells and fluorescence emission was measured on TECAN Infinite 200 fluorospectrophotometer. Bottom, HEK-OX1R cells were incubated with fluorescence probe (FluoForte) for 45 min. at 37° C. and then incubated with 1 μM of SB408124 for 1 h at 37° C. After pre-incubation, 1 μM of OxA was added to cells and fluorescence emission was measured.

FIG. 6: Determination of the inhibition of cellular growth of HEK-OX1R cells and colon adenocarcinoma cells (HT-29) induced by 0.1 μM of OxA or various concentrations of SB408124 antagonist. HEK-OX1R cells (black column) and HT-29 cells (white column) were incubated with 0.1 μM of OxA and indicated increasing concentration of SB408124, and cells were counted after 48 hr incubation. Results are expressed as the percentage of total viable cells.

FIG. 7: Effect of orexin-A and SB408124 antagonist on apoptosis in OX1R expressing colon adenocarcinoma cells, HT-29. HT-29 cells were challenged with 1 μM orexin-A or various concentration of SB408124 for 48 h. Apoptosis was measured by determination of annexin V-PE binding, and results are expressed as the percentage of apoptotic cells. Results are means±SE of three experiments. ***P<0.001.

FIG. 8: Effect of daily ip inoculation of OxA, Suvorexant and Almorexant on the length of colon from DSS- (dextran sulfate sodium) treated mice mimicking the acute ulcerative colitis disease. A. Mice were treated with 5% (w/v) DSS in drinking water and daily injected with 20 μg of OxA (OXA IP), 20 μg of Suvorexant (Suvo) or 20 μg of Almorexant (Almo). Control (wt) or treated DSS mice (DSS) were daily injected with 100 μl PBS. After one week of treatment, mice were sacrificed and the length of colon which represented a good marker of inflammation state, was measured. The figure displays a representative experiment. B. quantification of colon length from 5 different DSS-treated mice injected with OxA (DSS+OXA IP), Almorexant (DSS+Almo), Suvorexant (DSS+Suvo) or not injected (WT and DSS). NS, no significant; *, p<0.05; ** p<0.001

### EXAMPLES

### Example 1

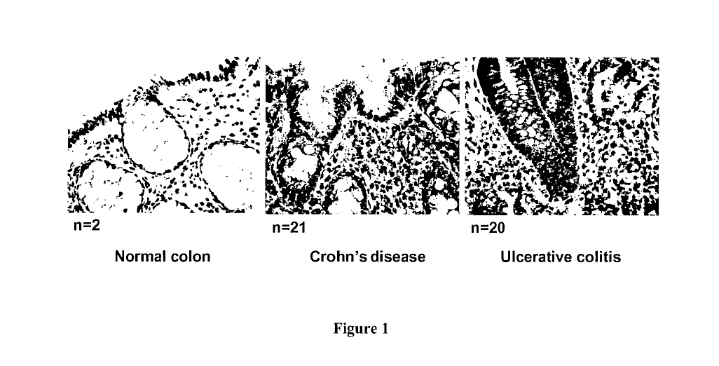

We demonstrate that OX1R was expressed in human Inflammatory Bowel Disease (IBD) including Crohn's disease and UC. Indeed, the use of specific antibodies directed against OX1R in immunohistochemistry (IHC) experiment of about 40 inflamed colonic samples revealed the ectopic presence of OX1R in both epithelial and immune cells (FIG. 1). In contrast OX1R was not expressed in normal colonic mucosa (FIG. 1).

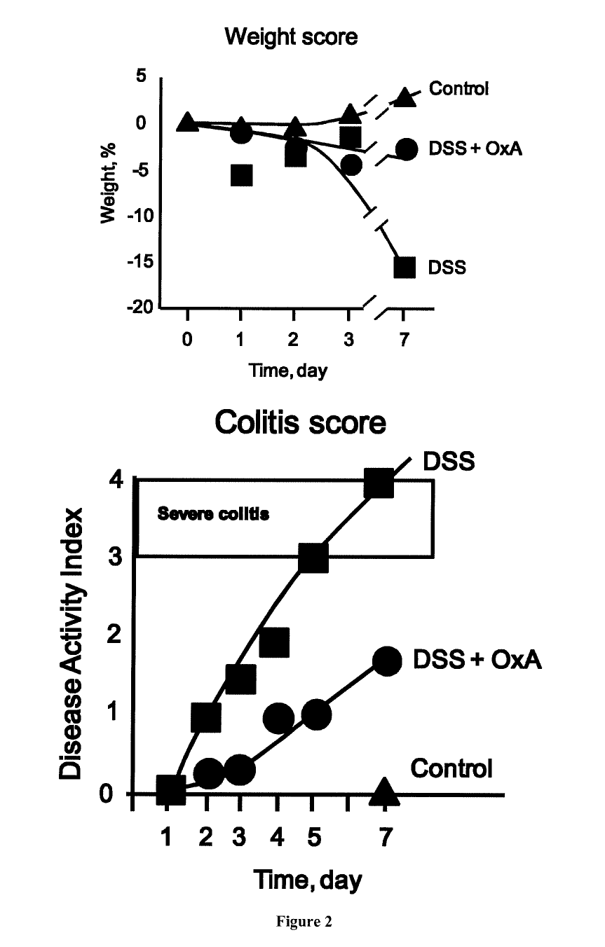

Based on these observations, we have investigated the effect of Orexin A (OxA) on acute inflammation in mice treated with Dextran Sulfate Sodium (DSS). OxA was preferentially chosen in these experiments since the presence of two disulphide bridges confers it more stability. DSS induces acute colitis characterized by weight loss, bloody diarrhea, intestinal ulcerations and infiltrations with granulocytes. Our results indicate that the treatment with OxA in orally DSS-treated mice ameliorates the Disease Activity Index (DAI) scored by measuring weight, length of colon, diarrhea and the presence of blood in the stool (FIG. 2).

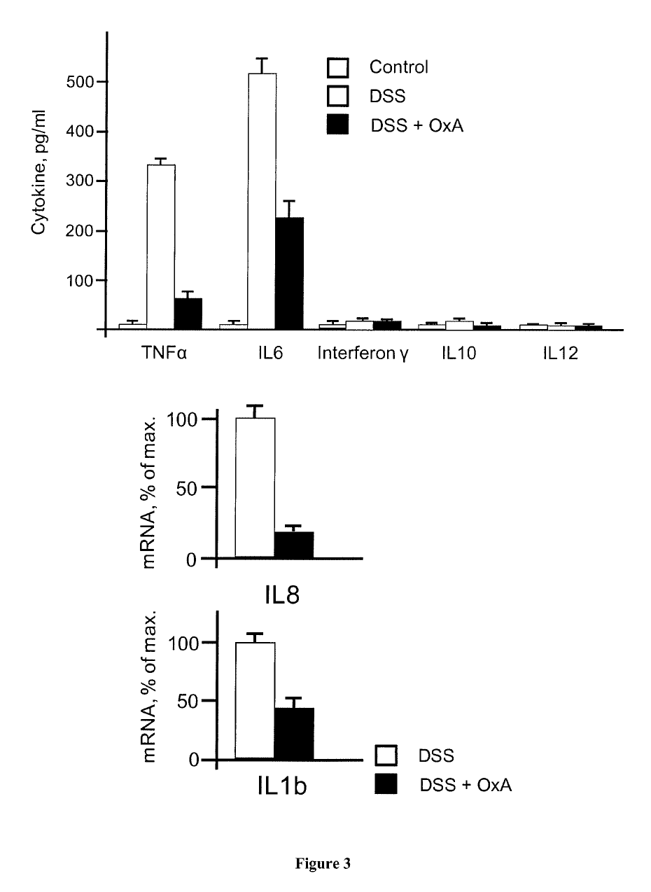

These observations are confirmed by histologic aspect of colon epithelium (histologic scoring by a GI pathologist). Thus, OxA has probably an anti-inflammatory effect on DSS-induced colitis. To confirm this hypothesis, we have investigated the anti-inflammatory effect of OxA treatment on cytokines secretion in colon extracts from DSS-treated mice. The analysis of cytokinic profile revealed that OxA reduces the secretion of “pro-inflammatory” cytokines such as TNFα, IL6, IL8 homo log and IL1B in colon extracts of DSS-induced colitis mice (FIG. 3). In contrast, OxA has no effect on INFγ, IL10, and IL12 cytokine secretion in colon extracts (FIG. 3).

DSS mouse model has been extensively used to decipher the mucosal inflammation in IBD, UC pathogenesis, and preclinical studies. However, mice show differential susceptibilities and responsiveness to DSS-induced colitis depending on their genetic background, gender. DSS model is a faithful model of inflammation but departs from Ulcerative Colitis in part for lack of the inflammation gradient from the rectum to the proximal colon usually observed in UC, any deregulation of ER stress in goblet cells known to be an early etiological factor of UC, and no digestive or extra-digestive complications of UC such as cholangitis and colorectal cancer, respectively. The use of genetically modified mice model represents a good alternative to chemically-induced colitis mice model. The recent development of a new spontaneous UC-like mouse model (EXCY2 mice, WO/2012/140516) consisting of a double KO of IL10 and Nox1 (IL10^{-/-}; Nox1^{-/-}) is ideally suited to study the OxA effect on spontaneous colitis. Indeed, EXCY2 mice 1) develop a spontaneous colitis at 6/7 weeks of age with an upwards gradient from the rectum and reproduce all molecular characteristics seen in UC including, loss of goblet cells, deregulation of ER stress, protective effect of tobacco; 2) spontaneously colonic cancer at 8 months of age (35-40% of mice); 3) develop mild cholangitis.

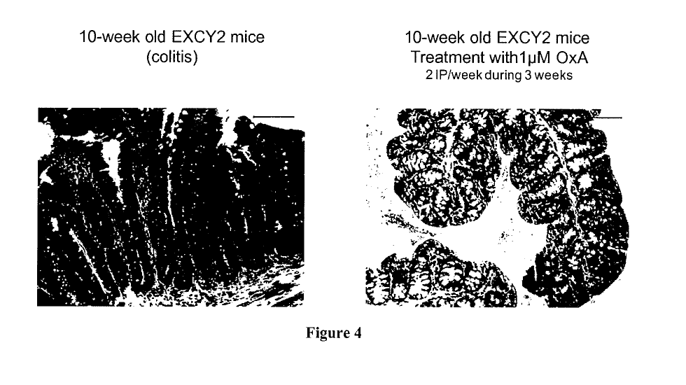

Our indicates that OxA treatment (0.22 μmoles/kg OxA, 2 IP/week during 3 weeks) alleviated severe colitis in 10 week-old EXCY2 mice, OxA-treated EXCY2 mice exhibited a normal colonic mucosa (general crypts aspect, crypt size, presence of goblet cells, absence of immune cells infiltration) as compared to vehicle treated EXCY2 mice (FIG. 4). Interestingly, we demonstrate that OxA/OX1R induced two main deregulated pathways involved in the onset of colitis (oxidative stress and ER stress) in both EXCY2 mice and Caco2 cells.

In conclusion these data indicate that 1) OxA could exert an original anti-inflammatory properties in DSS-treated mouse model; 2) OxA strongly protects from spontaneous colitis developed in EXCY2 mice model and trigger mucosal healing certainly by controlling the different pathways involved in the onset of UC. Taken into account this proof of concept, the system orexins/OX1R represent an innovative and effective target in the treatment of inflammatory bowel diseases, in particular ulcerative colitis.

### Example 2

Material & Methods

DSS-Induced Colitis Mice and EXCY2 Mice Models:

Balb/c mice were orally treated by 5% (w/v) of Dextran Sulfate Sodium (DSS) for 7 days. Orexin treatment was carried out in DSS treated mice by daily intraperitoneal injection of OxA (0.22 μmoles/kg) for 7 days. Weight and colitis symptoms (diarrhea, blood in the stool . . . ) for each mouse was daily measured. After 7 days of treatment, animals were sacrificed and colons were resected to further analyses (size, histological aspect, cytokine assays). EXCY3 mice (IL10^{-/-} and Nox1^{-/-}) which develop a spontaneous colitis at 6/7 weeks of age were treated or not with OxA (0.22 μmoles/kg, 2 intraperitoneal injection/week for 21 days). After animal sacrifice colons were resected and histologic analysis was performed.

Cytokine Assays:

Mice were sacrificed and colons were resected. Proteins were extracted from colon by tissue disruption in PBS using Tissue Lyser (Qiagen, Courtaboeuf, France). Various cytokines (see FIG. 3) were determined using Cytokine CBA kits (BD Sciences, Le Pont de Claix, France).

Ca2+ Mobilization Assay:

HEK cells expressing recombinant native OX1R (HEK-OX1R) cells were seeded in 96-wells plate, grown and maintained at 37° C. in a humidified 5% CO₂/air incubator. 80,000 cells/well were incubated with FluoForte probe according to FluoForte calcium assay kit (ENZO life Sciences, Farmingdale, N.Y., USA) for 45 min. at 37° C. and then incubated with or without 1 μM of 513408124 antagonist for 1 h at 37° C. After pre-incubation, 1 μM of OxA was added and fluorescence was determined using TECAN Infinite 200 fluorospectrophotometer.

Cells Growth Determination and Apoptosis Assay:

HEK-OX1R cells or colon adenocarcinoma HT-29 cells were seeded, grown and maintained at 37° C. in a humidified 5% CO₂/air incubator. After 24 hr culture, cells were treated with or without Orexin-A peptide or SB408124 antagonist, previously dissolved in DMSO, to be tested at the concentration indicated in the figure legends. After 48 hr of treatment, adherent cells were harvested by TriplE (Life Technologies, Saint Aubin, France) and manually counted. Apoptosis was determined using the Guava PCA system and the Guava nexin kit.

Results

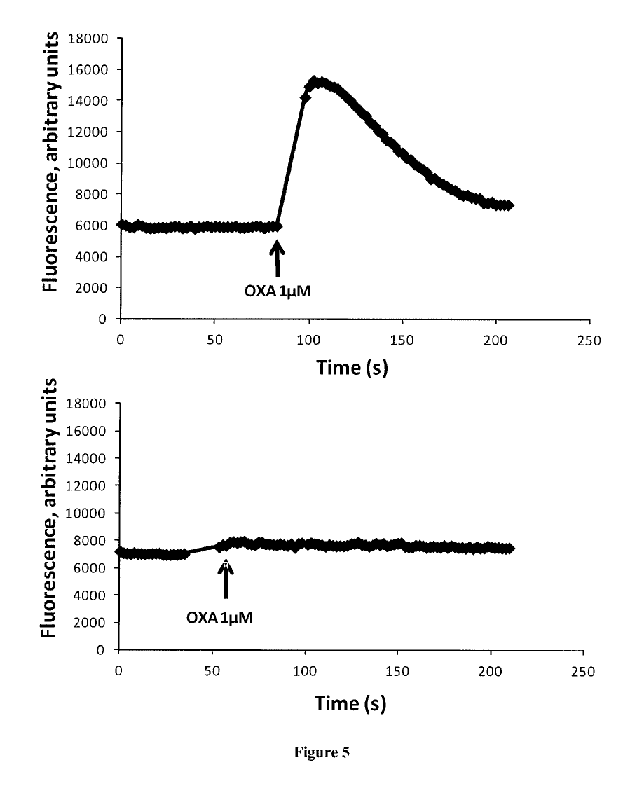

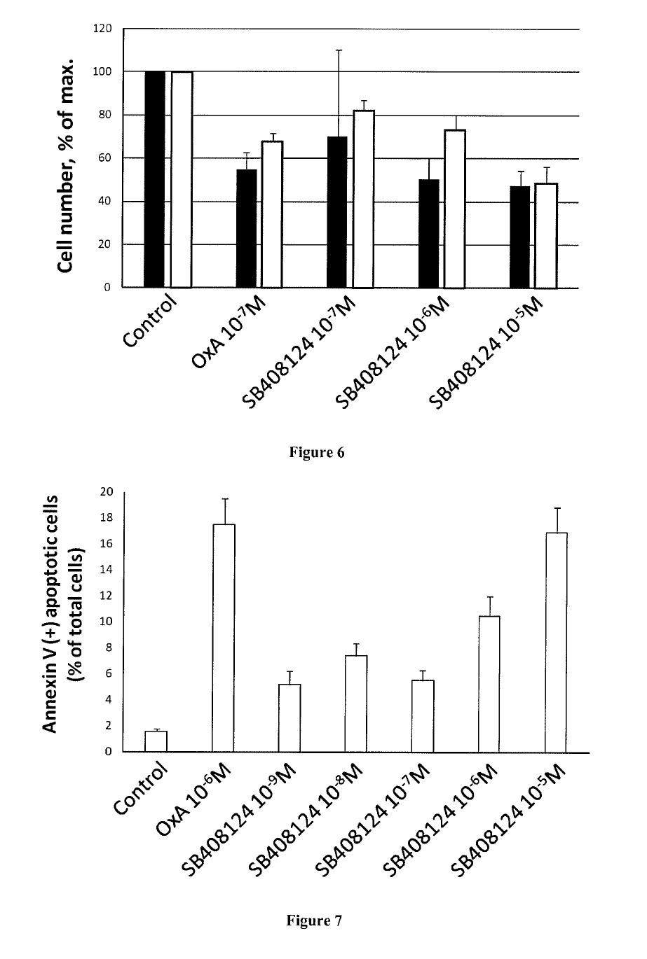

Without wishing to be bound to any particular theory, it is believed that the results observed in EXAMPLE 1 are due to the pro-apoptotic effects of Orexin. The inventors have now explored whether the OX1R antagonists of the prior art are capable to induce apoptosis. As shown in FIG. 4, OxA induced a large and transient Ca²⁺ mobilization in HEK-OX1R cells. In contrast, the preincubation of cells with 1 μM of SB408124 antagonist totally abolished the induced-Ca²⁺ mobilization (FIG. 4) confirming the antagonist effect of SB408124 on intracellular calcium release mediated by OX1R trough Gq and phospholipase C pathway. In the second phase, we determined the antagonist or agonist effect of SB408124 on cellular growth and apoptosis of HEK-OX1R cells and colon cancer cell line, HT-29. As shown in FIG. 5, OxA induced a strong inhibition of cellular growth of HEK-OX1R and HT-29 cells. Surprisingly, SB408124 antagonist induced also a strong inhibition in a dose-dependent manner of cellular growth of HEK-OX1R and HT-29 cells. As previously shown, orexins were able to trigger an inhibition of cellular growth by induction of mitochondrial apoptosis. As expected, OxA induced an apoptotic effect in HT-29 cells (FIG. 6). Likewise, SB408124 antagonist was also able to induce in a dose-dependent manner cell apoptosis in HT-29 cells. Taken together these results demonstrated that SB408124 was a full antagonist for OX1R-mediated calcium mobilization but a fill agonist for OX1R-mediated mitochondrial apoptosis in colon cancer cell line. The inventors now believe that such compounds could be suitable for the treatment of inflammatory bowel diseases.

### Example 3

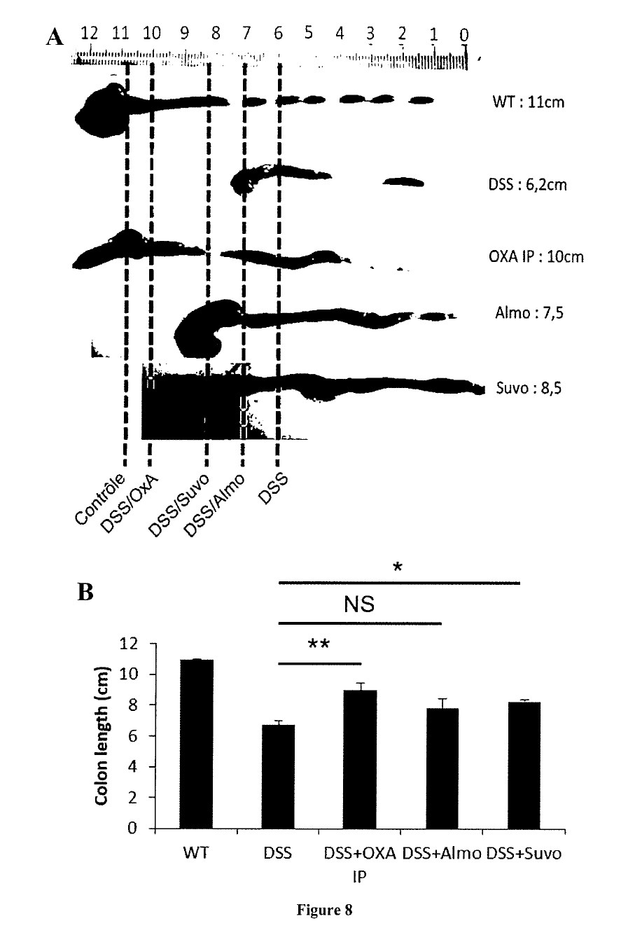

The inventors also investigated the effect of daily intraperitoneal inoculation of OxA, Suvorexant and Almorexant on the length of colon from DSS- (dextran sulfate sodium) treated mice mimicking the acute ulcerative colitis disease. As shown in FIGS. 8A and 8B, DSS treated mice show signs of acute ulcerative colitis disease as indicated by the diminution of colon length which represents a good marker of inflammation state. The inventors demonstrated that OX1R antagonists Suvorexant and Almorexant similarly to OxA protect mice from DSS induced acute ulcerative colitis disease (FIGS. 8A and 8B).

### REFERENCES

Throughout this application, various references describe the state of the art to which this invention pertains. The disclosures of these references are hereby incorporated by reference into the present disclosure.

1. A method of treating an inflammatory bowel disease in a subject in need thereof comprising administering to the subject a therapeutically effective amount of an OX1R antagonist.

2. The method of claim 1 wherein the subject suffers from ulcerative colitis, Crohn's disease, microscopic colitis, infectious colitis, radiation colitis, ischemic colitis, pediatric colitis, undetermined colitis, or functional bowel disorders.

3. The method of claim 1 wherein the OX1R antagonist is selected from the group consisting of SB408124, Suvorexant and Almorexant.

4. The method of claim 1 wherein the OX1R antagonist is administered to the subject in combination with a standard treatment selected from the group consisting of corticosteroids, immunosuppressive drugs, aminosalicylate sulfasalazines, Sulfasalazine, Balsalazide, Olsalazine, immunosuppressors and biological treatments.

5. The method of claim 2, wherein the Crohn's disease specifically affects the colon with or without ileitis.

6. The method of claim 2, wherein the microscopic colitis is lymphocytic colitis or collagenous colitis.

7. The method of claim 2, wherein the infectious colitis is caused by bacteria or by a virus.

8. The method of claim 2, wherein the aminosalicylate sulfasalazine is Mesalazine.

9. The method of claim 4, wherein the biological treatment is selected from the group consisting of Infliximab, Visilizumab, Adalimumab, Vedolizumab, golimumab and tofacitinib.
